(19) United States
(12) Patent Application Publication
(10) Pub. No.: US 2018/0330810 A1
(43) Pub. Date: Nov. 15, 2018

(54) PHYSICAL THERAPY MONITORING ALGORITHMS

(71) Applicant: Concorde Health, Inc., Boston, MA (US)

(72) Inventors: Viktor GAMARNIK, Brooklyn, NY (US); Guillaume DAVID, San Francisco, CA (US); Jeffrey VOGEL, Boston, MA (US)

(21) Appl. No.: 15/974,867

(22) Filed: May 9, 2018

Related U.S. Application Data
(60) Provisional application No. 62/537,214, filed on Jul. 26, 2017.
(60) Provisional application No. 62/503,564, filed on May 9, 2017.

(51) Int. Cl. G16H 20/30; G06T 7/246; A63B 24/00; A61B 5/00; G06F 3/16

(52) U.S. Cl. CPC G16H 20/30; G06T 7/251; A63B 24/0062; A63B 2024/0068; G06F 3/167; A61B 5/7405; A61B 5/7455; A61B 5/742

(57) ABSTRACT

Disclosed herein are systems and methods for providing guidance and support for physical therapy treatments.

### BACKGROUND OF THE INVENTION

As the population increasingly grows older, the number of people suffering from chronic, debilitating conditions resulting from heart attacks, strokes, and musculo skeletal injuries increases. Rehabilitative care and physical therapy can substantially treat these conditions and improve quality of life. Services and programs that provide guidance and feedback can enhance patient compliance and the success of physical therapy treatments.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, the invention provides a method for interpreting an exercise movement performed by a subject, the method comprising: a) receiving motion data from a motion tracking device based on the exercise movement performed by the subject; b) interpreting the motion data by the motion tracking device using a machine learning algorithm; c) determining by the motion tracking device whether the subject performed the exercise movement correctly based on a reference model of the exercise movement; and d) providing by the motion tracking device a recommendation to the subject to increase the accuracy of the exercise movement performed by the subject based on the reference model of the exercise movement.

In some embodiments, the invention provides a computer program product comprising a non-transitory computer-readable medium having computer-executable code encoded therein, the computer-executable code adapted to be executed to implement a method for interpreting an exercise movement performed by a subject, the method comprising: a) providing a physical therapy guidance system, wherein the physical therapy guidance system comprises: i) a data storage medium; ii) a detection module; iii) an accelerometer module; iv) a gyroscope module; v) a search module; vi) an interpretation module; and vii) an output module; b) storing by the data storage medium a reference model of the exercise movement; c) detecting by the detection module the exercise movement performed by the subject; d) searching by the search module the reference model of the exercise movement based on the exercise movement performed by the subject; e) measuring by the accelerometer module a speed of the exercise movement performed by the subject relative to the reference model of the exercise movement; f) measuring by the gyroscope module an orientation of the exercise movement performed by the subject relative to the reference model of the exercise movement; g) determining by the interpretation module whether the subject performed the exercise movement correctly based on the reference model of the exercise movement using machine learning; and h) outputting by the output module a recommendation based on the accuracy of the exercise movement performed by the subject.

### BRIEF DESCRIPTION OF THE DRAWINGS

FIG. 1 illustrates example displays of the present invention.

FIG. 2 illustrates a computer system for facilitation the methods, systems, or devices of the present disclosure.

FIG. 3 illustrates an example schematic of the user interface for onboarding a new user or an existing user of the physical therapy program.

FIG. 4 illustrates an example schematic of the user interface for general daily user flow.

FIG. 5 illustrates an example display of the onboarding process.

FIG. 6 illustrates an example display of the onboarding process.

FIG. 7 illustrates an example display of the onboarding process.

FIG. 8 illustrates an example display of the onboarding process.

FIG. 9 illustrates example displays of a communication interface between the user and a virtual AI physical therapy specialist.

FIG. 10 illustrates an example display of a communication interface between the user and a virtual AI-driven physical therapy specialist.

FIG. 11 illustrates an example display of communication interface between the user and a virtual physical therapy specialist.

FIG. 12 illustrates an example display of a communication interface between the user and a virtual physical therapy specialist.

FIG. 13 illustrates example displays of the user dashboard that summarizes a user's recovery process to provide an ongoing positive reinforcement resource.

FIG. 14 illustrates an example display of a video-based exercise regimen.

FIG. 15 illustrates an example display of a video-based exercise regimen communicated to the user by CUT.

FIG. 16 illustrates a schematic of the user interface for prevention user flow.

FIG. 17 illustrates a schematic of the user interface for challenge user flow.

FIG. 18 illustrates an example display of a challenge.

FIG. 19 illustrates an example display of a challenge.

FIG. 20 illustrates an example display of a challenge.

FIG. 21 illustrates an example display of a challenge.

FIG. 22 illustrates an example display of available raffles based on a rewards system.

FIG. 23 illustrates data segmentation of an exercise using a logistic regression model.

FIG. 24 illustrates filtered waveform motion data of an exercise using a logistic regression model.

FIG. 25 illustrates importance ranking of motion descriptors of a shoulder raise exercise using univariate selection analysis.

FIG. 26 illustrates importance ranking of motion descriptors of a shoulder raise exercise using recursive feature elimination.

FIG. 27 illustrates importance ranking of motion descriptors of a shoulder raise exercise using principle component analysis.

FIG. 28 illustrates the accuracy of various ranking methods for analyzing motion descriptors of a shoulder raise exercise.

FIG. 29 illustrates the precision of various ranking methods for analyzing motion descriptors of a shoulder raise exercise.

FIG. 30 illustrates the accuracy and precision using a logistic regression model for analyzing motion descriptors of a shoulder raise exercise.

FIG. 31 illustrates the accuracy and precision using a ridge regression model for analyzing motion descriptors of a shoulder raise exercise.

FIG. 32 illustrates the accuracy and precision using a linear discriminant analysis model for analyzing motion descriptors of a shoulder raise exercise.

FIG. 33 illustrates the accuracy and precision using a quadratic discriminant analysis model for analyzing motion descriptors of a shoulder raise exercise.

FIG. 34. illustrates the accuracy and precision using a Gaussian Naïve Bayes model for analyzing motion descriptors of a shoulder raise exercise.

FIG. 35 illustrates the accuracy and precision using a k-nearest neighbor model for analyzing motion descriptors of a shoulder raise exercise.

FIG. 36 illustrates the accuracy and precision using a support vector machine (SVM) RBF (radial basis function; Gaussian) kernel model for analyzing motion descriptors of a shoulder raise exercise.

FIG. 37 illustrates the accuracy and precision using a decision tree classifier model for analyzing motion descriptors of a shoulder raise exercise.

FIG. 38 illustrates the accuracy and precision using an extra tree classifier model for analyzing motion descriptors of a shoulder raise exercise.

FIG. 39 illustrates the accuracy and precision using random forest classifier model for analyzing motion descriptors of a shoulder raise exercise.

FIG. 40 shows the relative computation time of various classifier models.

### DETAILED DESCRIPTION OF THE INVENTION

Chronic, debilitating conditions resulting from heart attacks, strokes, and musculoskeletal injuries can negatively impact quality of life and contribute to the decline of physical function. Rehabilitative care and physical therapy can substantially treat these conditions and improve quality of life. Guidance that supports patient compliance can improve the success of physical therapy treatments and reduce the likelihood of further injuries.

A major factor to success of musculo skeletal injury rehabilitation is proper performance of physical therapy exercises. The disclosure provides a smart device that can be attached to a body part of a subject to track the movements of the body part and detect improper performance of an exercise. In some embodiments, the smart device can be attached to the subject by a one-size-fits-all band or strap. Non-limiting examples of attachment mechanisms include stretchable buckle, adjustable buckle, adjustable lock, a Velcro® mechanism, a single-use adhesive band, reusable adhesive band, or any combination thereof.

The device is not required to be rigidly or directly attached to the subject. In some embodiments, the device can be in the proximity of the subject and indirectly attached to the subject. For example, the device can be placed in a pocket of an article of clothing worn by the subject or held in the subject's hand.

In some embodiments, the device can be attached to a physical therapy equipment to track the movements of the equipment and detect improper performance of an exercise using the equipment. The device can be attached to equipment by a stretchable buckle, adjustable buckle, adjustable lock, a Velcro® mechanism, a single-use adhesive band, reusable adhesive band, or any combination thereof. Non-limiting examples of attachment mechanisms include a magnetic attachment, suction mount, static cling, Velcro® mechanism, and a combination thereof.

Musculoskeletal injuries can involve the damage of the muscular system, skeletal system, and vascular system. Musculoskeletal injuries are often caused by repetitive strenuous activity, such as postural strain, repetitive movements, overuse, and prolonged immobilization. Musculoskeletal conditions can affect the neck, clavical, hands, arm, knee, shoulders, pelvis, hip, and back. Non-limiting examples of musculoskeletal injuries can include compression, avulsion, separation, inflammation, and fracture.

Exercise that includes muscle strengthening, stretching, aerobic conditioning, or a combination thereof can help alleviate pain and rehabilitate musculoskeletal injuries. For example, lower back injuries can be treated with specific exercises that strengthen the low back muscles and abdominal muscles that help support the back muscles. Abdominal strengthening exercises can include, for example, sit-ups, crunches, abdominal machines, and leg raises. Low back exercises can include, for example, exercises performed on machines, and by simply lying on the stomach and slowly raising the chest off the ground. These exercises utilize the lower back muscles to stretch the spine. Shoulder exercises can include, for example, high/low rows, reverse chest flys, pendulums, wall climbers, rows, wall angels, thoracic rotations, Prone T's, and Prone scapular retractions. Cervical spine exercises can include, for example, upper trapezius stretch, levator stretch, upright rows, wall angels, Prone Y's, and Prone T's. Knee exercises can include, for example, quadriceps sets, heel slides, clamshells, and tap downs. Ankle exercises could include, for example, heel raises, toe raises, star drills, and piriformis stretch.

The present invention provides a portable and convenient method to facilitate physical therapy exercises both in the physical therapist's setting and the home. For example, a physical therapist can demonstrate an exercise to a patient in-person while using the present invention. The physical therapist provide recommendations to the patient including, for example, the proper form to maintain during performance of an exercise, the orientation of an exercise, the speed of an exercise, the number of repetitions of an exercise, and any precautionary alerts.

In some embodiments, the physical therapist can input to the present invention a prescribed exercise and associated parameters for the patient to perform at home. Associated parameters can be based on predetermined parameters of the patient including, for example, the type of injury; the severity of the injury; and biological parameters, such as weight, height, body habitus, body dimensions, and age.

Successful physical therapy interventions rehabilitate both the physical and psychological aspects of recovery. The present invention can provide a personalized daily timeline of exercise and tracks the progress. Progress tracking can include logging compliance with a scheduled regimen by time- and date-stamping motion data during recordation. Non-limiting examples of progress tracking can include information about whether the patient performs the exercises during the correct times, whether the patient skips any scheduled sessions, whether certain exercises are performed out of order or too close together in time, adequacy of the performance, patient compliance to the feedback provided by the device, and patient experience before, after, and during an exercise (e.g. comfort/discomfort or ease/difficulty).

The present invention allows a patient to perform rehabilitative exercises at home with the guidance and support provided by the present invention between doctor visits.

In some embodiments, patient data and analyses of patient data can be uploaded to the server for storing in an electronic health record. In some embodiments, patient data can be communicated to an external device for data analysis. In some embodiments, the present invention can provide a medium for the patient to communicate with the physical therapist and/or other support specialists.

In some embodiments, the present invention can provide visual demonstrations of exercises, for example, in the form of images and/or video. The present invention can further provide audial (audio) instructions and constructive feedback to inform the patient and provide psychological support throughout the physical rehabilitation process. In some embodiments, the present invention can provide haptic or tactile feedback, for example, through vibrations of the motion tracking device. In some embodiments, the present invention can inform the user through haptic or tactile notifications. For example, the pace of an exercise can be communicated to the user by frequency and repetitions of a vibration. To reduce the likelihood of overextension or underextension of a resistance exercise, the device can vibrate at the upper limit and the lower limit of the exercise. The present invention can provide personalized, step-by-step guidance to the user during an exercise to reduce the likelihood of improper form and additional injury.

In some embodiments, the present invention can provide features that engage the user and enhance the user experience including, for example, educational trivia and a rewards system. A rewards system can encourage patient compliance and reduce return-to-work times, for example, after an occupational injury.

### Calibration

Motion tracking and interpretation thereof can be improved by proper calibration of the device. Calibration of the present invention can depend on where the motion tracking device is attached. For example, the device can be directly attached to a subject or the device can be attached to a physical therapy equipment. For example, the device can be attached to a specific limb of a subject, or a specific part of the physical therapy equipment. Calibration of the device can further depend on the orientation of the motion tracking device. For example, the orientation of the device can be described with respect to gravity, a body part, or a piece of equipment.

In some embodiments, the device is in a general orientation. For example, the device is on the left forearm of a subject with the screen of the device facing away from the skin and the power button adjacent to the wrist. In some embodiments, the description of the orientation of the device can be specific. For example, the device is on the ventral aspect of the left forearm, halfway between the styloid process of the radius and the lateral epicondyle of the elbow, the screen facing away from the skin, and the power button facing toward the elbow and away from the wrist.

In some embodiments, calibration can be based on patient health information. Non-limiting examples of patient health data include height, weight, body habitus, specific body dimensions, and the type and severity of injury. In some embodiments, calibration can require information about the exercise equipment. Non-limiting examples of information about the exercise equipment include the type of equipment and the model name.

Calibration of the motion tracking device can be based on baseline orientation and movement data from the user. For example, the user can be required to maintain a certain position or series of positions for several seconds. In some embodiments, the user can be required to perform a certain motion or series of motions to provide baseline information. This information can be generated independently by the user or with assistance from the physical therapist and/or physician.

Calibration data can be generated, for example, only once at the beginning of the engagement with a physical therapy routine by a user. In some embodiments, calibration data can be generated before each exercise, at regular time intervals throughout the exercise, or when the software detects a need for re-calibration.

To reduce the likelihood of injuries, calibration data can include specific upper limit and lower limit orientations for each exercise to enhance accuracy of movements. For example, to reduce the likelihood of a shoulder injury during extended arm abduction exercises, a specific calibration data point can be when the arm is positioned at or close to 90 degrees of abduction.

In some embodiments, the present invention can measure and track biological parameters of the patient during performance of an exercise. Non-limiting examples of biological parameters can include heart rate, respiratory rate, sweating, and oxygen saturation. Biological parameters can be measured through various sensors of the device, including, for example, a camera.

### Motion Tracking & Data Analysis

The present invention includes a motion tracking device that identifies an exercise movement or a series of exercise movements initiated by a user. In some embodiments, the motion tracking device is a mobile telecommunications device. Non-limiting examples of a mobile telecommunications device include a smart phone, a smart watch, a personal digital assistant (PDA), a tablet, an e-book (e-reader or e-book reader device), a gaming device, a media player, and any wearable smart device. The motion tracking device can include various sensoring features and modules that detect motion pertaining to velocity, angle, direction, and range. For example, the motion tracking device can include an accelerometer that detects the orientation, speed, and acceleration of an exercise movement; a gyroscope that detects the orientation and angular velocity of the exercise movement; and a magnetometer that detects the strength and direction of a magnetic field. In some embodiments, the motion tracking device can include, for example, a camera to detect visual activity and a microphone to detect audial (audio) activity.

The motion tracking device can be configured to assess exercise movement based on input data, including, for example, calibration data, a prescribed or reference model of an exercise movement, patient-specific guidelines, historical or pre-existing compliance and performance data, and motion sensor data.

Motion sensor data can be used to estimate the orientation of the motion tracking device at each time point during use. The motion data information can be combined with user input information that describe where and how the device is attached to the user or exercise equipment and user-specific parameters to estimate the orientation of the motion tracking device to the body part and/or the exercise equipment.

In some embodiments, the motion tracking device can include sensors that track daily mobility and biological parameters of the subject to provide a comprehensive health profile during a physical rehabilitation process. Non-limiting examples of biological parameters can include blood pressure, body temperature, heart rate, respiratory rate, blood alcohol content, blood glucose, electrodermal activity, sweating, forced expiratory volume per second, forced vital capacity, oxygen saturation levels, and perfusion index. Non-limiting examples of daily mobility parameters include number of steps taken, number of flights climbed, walking and running distance, and number of calories burned. For example, the motion tracking device can include a pedometer that counts the number of steps taken by detecting the motion of the hands or the hips.

In some embodiments, the motion and orientation data can be amalgamated from multiple sensors using sensor-fusion techniques to reduce the amount of uncertainty regarding the accuracy of an exercise movement compared to data acquired from sensors individually. Sensor-fusion techniques can be used to interpret signals arising from simple motions (uniaxial) and complex motions (multiaxial). Depending on the type of exercise and the location of injury, the exercise motion can be simple or complex. Uniaxial motion can include sliding or gliding movements. For example, a bicep curl involves uniaxial up and down movements at the elbow hinge joint. Biaxial motion can include movements on two planes or axes. For example, a side-to-side movement (adduction and abduction) and forward and backward movement (flexion and extension) of the hand at the wrist joint. Multiaxial motion can include movements on two or more planes or axes. For example, a shoulder rotation involves multiaxial rotation of the shoulder joint in all directions.

In some embodiments, accelerometer signals can be combined with gyroscope signals to improve the accuracy of exercise movement tracking. Signals from each sensor can be filtered to improve the quality and signal-to-noise ratio (SNR). For example, accelerometer data can be passed through a moving-average filter to reduce noise. Other SNR filtering methods include filters that operate in the stationary wavelet domain.

The present invention can process and interpret motion data using a variety of algorithms including, for example, machine learning, supervised learning, unsupervised learning, reinforcement learning, decision tree, decision tree classifier, extra tree classifier, random forest classifier, naïve Bayes classification, Gaussian naïve Bayes, Bayesian inference, hidden Markov models (HMM), clustering, support vector machine (SVM), radial basis function (RBF) kernel, neural network, deep neural network, linear regression, linear discriminate analysis, quadratic discriminate analysis, logarithmic regression, logistic regression, ridge regression, k-nearest neighbors (k-NN), k-means clustering, random forests or random decision forests, dimensionality reduction, gradient boosting, and adaptive boosting. In some embodiments, the present invention can use various selection methods including, for example, univariate selection analysis, recursive feature elimination, and principle component analysis. For example, the present invention can provide an artificial intelligent (AI) specialist that communicates with the user to accomplish tasks, including, for example, to manage the onboarding process, monitor user recovery process, provide therapy recommendations, and communicate user information with health care professionals.

In some embodiments, clustering of motion data can be performed using the k-nearest neighbors algorithm. This algorithm can be used to group patients based on similarities with respect to certain criteria. The motion data estimation algorithm can be tuned according to which group the patient belongs.

In some embodiments, a SVM or a regression analysis can be used to classify whether a patient is performing a movement that is beneficial to recovery or detrimentral to recovery. For example, user motions and sequential orientations can be classified using SVM that was previously trained on previously acquired data from the actual user and other individuals. The present invention provides a guidance to reduce the likelihood of further injury to the patient during the physical rehabilitation process.

The present invention includes a real-time motion data feed that can be displayed to the user. The user can visually adjust exercise movements based on the consistency of the motion data. The user can also review the motion data to determine the timing and consistency of errors. The present invention can analyze and compare the motion data to determine the cause of an erroneous movement. Accordingly, the present invention can inform the user of the error, how to fix the error, and provide immediate feedback to the subject. The motion data can also be uploaded or communicated to another device. A physical therapy specialist can also review the motion data and provide additional feedback to the subject.

In some embodiments, the present invention includes a live-imaging feature that records exercise movements of the subject through a camera module and/or a video module. A pictorial representation can be displayed on the telecommunications device to mimic the real-time exercise movements of the user. In some embodiments, the pictorial representation can be used to display a demonstration of an exercise.

In some embodiments, the present invention includes a voice feature. The voice feature can provide audible status updates to the user so that the user is not required to view the display during performance of the exercise.

Non-limiting examples of motion parameters include range of motion, velocity of motion, duration of sustained positions, number of exercise repetitions, and number of exercise sets. In some embodiments, motion parameters can be estimated using a neural network trained on previously acquired patient data. Training data can be obtained from the actual user, or can be generated from a collection of users who have similar parameters as the actual user. For example, a collection of users can share similar height, weight, age, build, frame, injury type, and injury severity.

Analysis of motion and orientation data from the user can be compared to a template or a set of known desired values (target value). If the mismatch between the desired orientation and the actual orientation exceeds a pre-determined cutoff value, then the user is informed of the mismatch and offered feedback on how to reduce the mismatch.

In some embodiments, adequacy of performance can be judged using dynamic cutoffs or templates, which can be adjusted based on previous and/or current performance. For example, the target range of motion of a user can increase over time as performance continually improves. Alternatively, the target range of motion of a user can be temporarily reduced if a user is detected to be missing the target motion due to pain or discomfort.

The present invention can interpret pain, discomfort, or strain, for example, by detecting reduced acceleration of a motion by the user before the range of motion reaches the target motion of an exercise. In some embodiments, the user can temporarily stop the exercise to take a rest break and/or continue the exercise with a shorter range of motion.

### Recommendation

The present invention provides a method to support physical therapy rehabilitation by detecting and interpreting an exercise movement or a series of exercise movements by a user. Feedback from the device can be provided by audio, visual, and tactile/haptic means. For example, the user can receive audio alerts that encourage and reinforce good performance or provide warnings and corrective advice poor performance. Audio alerts can be simple tones and/or pre-recorded voice prompts. For example, different tones and/or voice prompts can be played when optimal or sub-optimal movements are detected, or during the end and the beginning of an exercise set.

In some embodiments, the feedback can be provided by haptic means. For example, different vibration patterns of the device can alert the user if the detected motion has exceeded the recommended range of motion, or during the end and/or the beginning of an exercise.

In some embodiments, the feedback can be provided by visual means through a display screen of the device. The display can display the detected exercise regimen. In some embodiments, the user can confirm the detected exercise regimen or cancel an incorrectly detected exercise regimen. In some embodiments, the user can manually select a reference model of an exercise movement.

In some embodiments, audio and visual feedback can be transmitted to a peripheral or external device. For example, the device can be connected to a personal computer, a television, or a speaker system. Feedback alerts can be provided by a peripheral or external device that is communicatively coupled to the device. Reference models of exercise movements can be input from a peripheral or external device that is communicatively coupled to the server.

Feedback can be provided in a delayed fashion, real-time during, before, and after performance of an exercise regimen. For example, feedback and recommendations can be provided to the user prior to performing the exercise to improve form based on previous performance. In some embodiments, feedback can be provided in real-time as the user is performing the exercise. The present invention can guide a patient towards optimizing form and promoting compliance to decrease overall recovery time. Automatic, immediate feedback can reduce the likelihood of injury. If the motion tracking device detects a high risk of injury, the prescribing physician or physical therapist can be informed to intervene.

### Computer Architectures

One aspect of the disclosure provides a system that is programmed or otherwise configured to implement the methods of the disclosure. The system can include a computer server that is operatively coupled to a mobile telecommunications device.

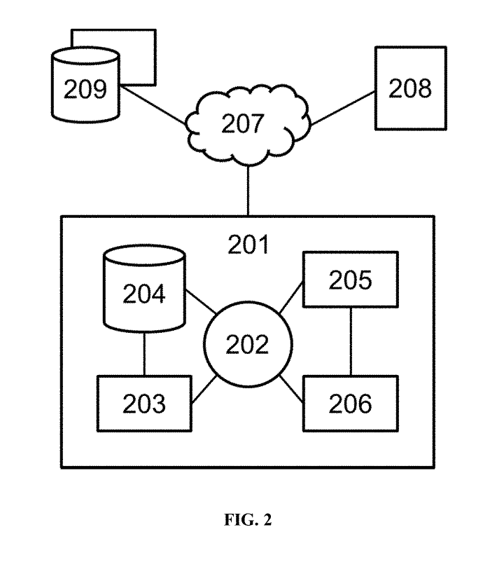

FIG. 2 shows a computer system programmed or otherwise configured to allow, for example, detection of an exercise movement by a user and interpretation of the exercise movement relative to a reference model of the exercise movement. A user can view a variety of reference models of an exercise movement and manually select a desired reference model. In some embodiments, a user can manually create and add a new exercise regimen.

The system of FIG. 2 includes a computer server (“server”) 201 that is programmed to implement methods disclosed herein. The server 201 includes a central processing unit (CPU) 202, which can be a single core or multi-core processor, or a plurality of processors for parallel processing. The server 201 also includes a memory 203, such as random-access memory, read-only memory, and flash memory; an electronic storage unit 204, such as a hard disk; a communication interface 205, such as a network adapter, for communicating with one or more other systems; and peripheral devices 206, such as cache, other memory, data storage, and electronic display adapters. The memory 203, storage unit 204, interface 205, and peripheral devices 206 are in communication with the CPU 202 through a communication bus, such as a motherboard. The storage unit 204 can be a data storage unit or data repository for storing data. The server 201 can be operatively coupled to a computer network 207 with the aid of the communication interface 205. The network 207 can be the Internet, an internet or extranet, or an intranet or extranet that is in communication with the Internet. In some embodiments, the network 207 is a telecommunications network or data network. The network 207 can include one or more computer servers, which can allow distributed computing, such as cloud computing. In some embodiments, the network 207, with the aid of the server 201, can implement a peer-to-peer network, which can allow devices coupled to the server 201 to behave as a client or an independent server.

The storage unit 204 can store files, such as drivers, libraries, saved programs, and clinical data related to a subject. The storage unit 204 can store clinical data from, for example, EMRs and EHRs. The storage unit 204 can store motion data and other subject data, such as biometric data, physiological readings, vital signs, body weight, and treatments at various points of a subject's medical history. The server 201 in some cases can include one or more additional data storage units that are external to the server 201, such as located on a remote server that is in communication with the server 201 through an intranet or the Internet.

The server 201 can communicate with one or more remote computer systems through the network 207. In some embodiments, the server 201 is in communication with a first computer system 208 and a second computer system 209 that are located remotely with respect to the server 201. The first computer system 208 can be the computer system of a user, and the second computer system 209 can be an external data repository. The first computer system 208 and second computer system 209 can be, for example, personal computers, such as a portable PC or laptop; slate and tablet PC, such as Apple® iPad and Samsung® Galaxy Tab; telephones; smartphones, such as Apple® iPhone, Android-enabled device, Windows® Phone, Blackberry®, Google® Pixel, Amazon® Fire Phone and; smart watches, such as Apple® Watch; smart glasses, such as Google® Glass; or personal digital assistants. The user can access the server 201 via the network 207 to use the invention.

In some embodiments, the system includes a single server 201. In other situations, the system includes multiple servers in communication with one another through an intranet or the Internet. The server 201 can be adapted to store exercise regimens, exercise information, user information, and other information of relevance to exercise regimens and the user. Exercise and user information can be stored on the storage unit 204 of the server 201.

Methods as described herein can be implemented by way of a machine- or computer-executable code or software stored on an electronic storage location of the server 201, such as, for example, on the memory 203 or electronic storage unit 204. During use, the code can be executed by the processor 202. In some embodiments, the code can be retrieved from the storage unit 204 and stored on the memory 203 for ready access by the processor 202. In some embodiments, the electronic storage unit 204 can be precluded, and machine-executable instructions are stored on memory 203. Alternatively, the code can be executed on the second computer system 209. The code can be pre-compiled and configured for use with a processor adapted to execute the code, or can be compiled during runtime. The code can be supplied in a programming language that can be selected to allow the code to execute in a pre-compiled or as-compiled fashion.

All or portions of the software can at times be communicated through the Internet or various other telecommunications networks. Such communications can support loading of the software from one computer or processor into another, for example, from a management server or host computer into the computer platform of an application server. Another type of media that can bear the software elements includes optical, electrical, and electromagnetic waves, such as those used across physical interfaces between local devices, through wired and optical landline networks and over various air-links. The physical elements that carry such waves, such as wired or wireless links, or optical links, also can be considered as media bearing the software.

A machine-readable medium, incorporating computer-executable code, can take many forms, including a tangible storage medium, a carrier wave medium, and physical transmission medium. Non-limiting examples of non-volatile storage media include optical disks and magnetic disks, such as any of the storage devices in any computer, such as can be used to implement the databases of FIG. 2. Volatile storage media include dynamic memory, such as a main memory of such a computer platform. Tangible transmission media include coaxial cables, copper wire and fiber optics, including wires that comprise a bus within a computer system. Carrier-wave transmission media can take the form of electric or electromagnetic signals, or acoustic or light waves such as those generated during radio frequency (RF) and infrared (IR) data communications.

Common forms of computer-readable media include: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, and any other medium from which a computer can read programming code or data.

Many of these forms of computer readable media can be involved in carrying one or more sequences of one or more instructions to a processor for execution.

The server 201 can be configured for: data mining; extract, transform and load (ETL); or spidering operations, including Web Spidering where the system retrieves data from remote systems over a network and access an Application Programming Interface or parses the resulting markup, which can permit the system to load information from a raw data source or mined data into a data warehouse. The data warehouse can be configured for use with a business intelligence system, such as Microstrategy® and Business Objects®. The system can include a data mining module adapted to search for media items in various source locations, such as email accounts and various network sources, such as social networking accounts, such as Facebook®, Foursquare®, Google+®, and LinkedIn®, or on publisher sites, such as weblogs.

Computer software can include computer programs, such as, for example executable files, libraries, and scripts. Software can include defined instructions that upon execution instruct computer hardware, for example, an electronic display to perform various tasks, such as display graphical elements on an electronic display. Software can be stored in computer memory.

Software can include machine-executable code. Machine-executable code can include machine language instructions specific to an individual computer processor, such as a CPU. Machine language can include groups of binary values signifying processor instructions that change the state of an electronic device, for example, a computer, from its preceding state. For example, an instruction can change the value stored in a particular storage location inside the computer. An instruction may also cause an output to be presented to a user, such as graphical elements to appear on an electronic display of a computer system. The processor can carry out the instructions in the order they are provided.

Software comprising one or more lines of code and their output(s) can be presented to a user on a user interface (UI) of an electronic device of the user. Non-limiting examples of UIs include a graphical user interface (GUI) and web-based subject interface. A GUI can allow a subject to access a display of the invention. The UI, such as GUI, can be provided on a display of an electronic device of the user. The display can be a capacitive or resistive touch display, or a head-mountable display, such as a Google® Glass. Such displays can be used with other systems and methods of the disclosure.

Methods of the disclosure can be facilitated with the aid of applications, or apps, which can be installed on an electronic device of the subject. An application can include a GUI on a display of the electronic device of the subject. The application can be programmed or otherwise configured to perform various functions of the system. GUIs of apps can display on an electronic device of the subject. Non-limiting examples of electronic devices include computers, televisions, smartphones, tablets, and smart watches. The electronic device can include, for example, a passive screen, a capacitive touch screen, or a resistive touch screen. The electronic device can include a network interface and a browser that allows the subject to access various sites or locations, such as web sites, on an intranet or the Internet. The application is configured to allow the mobile device to communicate with a server, such as the server 201.

Any embodiment of the invention described herein can be, for example, produced and transmitted by a user within the same geographical location. A product of the invention can be, for example, produced and/or transmitted from a geographic location in one country and a user of the invention can be present in a different country. In some embodiments, the data accessed by a system of the invention is a computer program product that can be transmitted from one of a plurality of geographic locations to a user. Data generated by a computer program product of the invention can be transmitted back and forth among a plurality of geographic locations, for example, by a network, a secure network, an insecure network, an internet, or an intranet. In some embodiments, a system herein is encoded on a physical and tangible product.

The present invention comprises a computer program product comprising a non-transitory computer-readable medium having computer-executable code encoded therein that is adapted can be executed to interpret an exercise movement performed by a subject. The method provides a physical therapy guidance system that can include a data storage medium, a detection module, an accelerometer module, a gyroscope module, a magnetometer module, a search module, a display module, and an output module.

The data storage medium can store exercise regimens, user health data, and instructions to execute functions performed by the detection module, the accelerometer module, the gyroscope module, the magnetometer module, the search module, the display module, and the output module of the physical therapy guidance system.

The detection module detects an exercise movement performed by the subject. The detection module continuously tracks the exercise movement through optical and tactile sensors to synthesize motion data. The motion data can then be communicated to various modules on the motion tracking device or a remote, external device for processing and data analysis. Transmission of data to a remote device can be carried out through a GSM/CDMA network, local WiFi, Bluetooth, or any wireless communication technology over a secure connection.

For example, the search module can process the motion data to detect a reference model of an exercise movement that the user intends to perform. The search module can be communicatively coupled to a predictive module that calculates the probability of a specific exercise regimen that the user is performing or desires to perform based on historical factors, such as use history, search history, and patient health information.

The motion data can be further analyzed by an interpretation module, which can interpret incorporated motion data tracked by the accelerometer module, the gyroscope module, and the magnetometer module. The accelerometer module can measure the speed and acceleration of the exercise movement performed by the subject. The accelerometer module can further compare the speed and acceleration of the exercise movement relative to a reference model of the exercise movement. The gyroscope module can measure the orientation of the movement performed by the subject. The gyroscope module can further compare the orientation of the movement relative to a reference model of the exercise movement. The magnetometer module can detect the strength and direction of a magnetic field and helps orient and accurately measure the positioning of the motion tracking device. The interpretation module can interpret incorporated motion data and determine whether the subject performed the exercise movement correctly based on a reference model of the exercise movement by machine learning algorithms.

The output module can output and provide a recommendation to the user based on the accuracy of the exercise movement performed by the subject. For example, if the acceleration of the exercise movement is faster than the recorded speed of the reference model of the exercise movement, the output module can output an error alert notification and a corresponding suggestion to the user to rectify the mistake. For example, the alert notification and corresponding suggestion can be “ALERT! Motion is too fast. Try reducing the speed.” The alert notification can be displayed visually by the display module and/or by an audible or haptic notification.

Based on the analysis of the motion data, a reference model of the exercise movement can be displayed by the display module. The display module can display visual demonstrations of a reference model of the exercise movement.

### EXAMPLES

### Example 1: Physical Therapy Program for Rehabilitating a Leg Injury

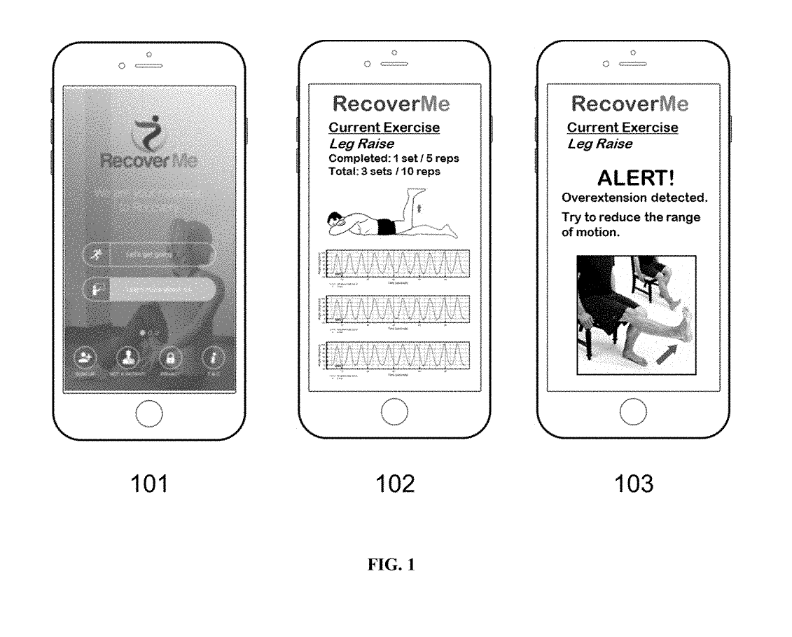

FIG. 1 illustrates example displays of the present invention on a mobile communications device. 101 illustrates an example home login page of the application, which requires patient login information to access confidential patient information and personalized exercise regimens. 102 illustrates an example display during performance of a leg raise exercise. The display shows the current exercise being performed, the number of sets and repititions of the exercise regimen, a pictorial demonstration of the exercise, and a plurality of motion tracking data. In some embodiments, the display can include a progress icon for the specific exercise, a progress icon for the overall physical therapy regimen. 103 illustrates an example display during detection of an improper motion. The display can alert the user that an improper motion has been detected, for example, an overextension that is characterized by a range of motion that exceeds an upper limit. The display can also provide a recommendation on how to correct the improper motion, for example, by reducing the range of motion.

### Example 2: Physical Therapy Program for Rehabilitating a Shoulder Injury

A patient can be diagnosed with a shoulder injury and experiences pain over the top of the shoulder or down the outside of the arm, shoulder weakness, and reduced mobility of the shoulder. Non-limiting examples of shoulder injuries include shoulder impingement, shoulder strain, and shoulder sprain.

The physical therapist and physician can prescribe exercises to strengthen and rehabilitate the patient's rotator cuff muscles by prescribing a reference model of an exercise movement in the present invention. The patient can perform the exercise using the present invention at the doctor's office or at home. The present invention can prompted the user to specify patient parameters to personalize the exercise to the user.

Alternatively, a personalized exercise regimen devised by the physical therapist or physician on an external device can be provided to the patient electronically and the personalized exercise regimen can be downloaded onto a smart device to run the personalized exercise regimen. A personalized exercise regimen can incorporate specific patient and injury parameters that can be determined and set by the health professional. For example, the upper limits of the exercise can be set on the personalized exercise regimen to reduce the likelihood of hyperextension and additional injury during performance of the exercise. The upper limits can be based on a physical and health assessment of the patient by the health professional. For example, the exercise can be modified based on the degree of injury, the pain tolerance of the patient, and the strength of the injured area.

Through various machine learning algorithms, the present invention can determine the pain tolerance and track the recovery progress of the patient. For example, incomplete movements of an exercise can be interpreted as pain and/or inability to complete the exercise. Accordingly, the present invention can modify the exercise to reduce the range of motion of the exercise until the patient is able to complete the exercise properly and consistently. The present invention provides real-time analysis of patient progress to personalize the physical therapy rehabilitation process to the patient.

The present invention can also provide recommendations or reminders prior to, during, or after performance of the exercise. For example, the present invention can provide advice on how to improve posture and form while performing the exercise. In addition, the present invention can provide educational trivia to the patient to reduce the likelihood of exacerbating the injury or cause re-injury. For example, the patient can be advised to avoid specific everyday activities that can induce re-injury.

### Example 3: Physical Therapy Program for Rehabilitating a Spinal Cord Injury

A patient can be diagnosed with a spinal cord injury and experiences pain on various part of the body, such as wrist, elbow, back, shoulder, knee, hip, hand, foot, and cervix. Non-limiting examples of a spinal cord injury include cervical spine injury, paraspinal muscle injury surrounding or in proximity to the cervical spine, thoracic spine injury, lumbar spine injury, sacral spine injury, and sacral spine herniation injury.

The physical therapist and physician can prescribe exercises to strengthen and rehabilitate the injured muscles by prescribing a reference model of an exercise movement in the present invention. The present invention can prompted the user to specify patient parameters to personalize the exercise to the user. The user can specify other locations of injury and/or immobility.

### Example 4: Physical Therapy Program for Rehabilitating an Elbow Injury

A patient can be diagnosed with an elbow injury and experiences pain on the elbow and wrist, and weakened grip strength. Non-limiting examples of elbow injuries include tennis elbow, lateral epicondylitis, medial epicondylitis, and elbow strain.

The physical therapist and physician can prescribe exercises to rehabilitate the muscles around the elbow and strengthen the muscles of the forearm by prescribing a reference model of an exercise movement in the present invention. Non-limiting examples of elbow rehabilitative exercises include wrist rotations, wrist extensions, wrist flexion, and bicep curls. The present invention can prompted the user to specify patient parameters to personalize the exercise to the user.

### Example 5: Physical Therapy Program for Onboarding New Users or Existing Users

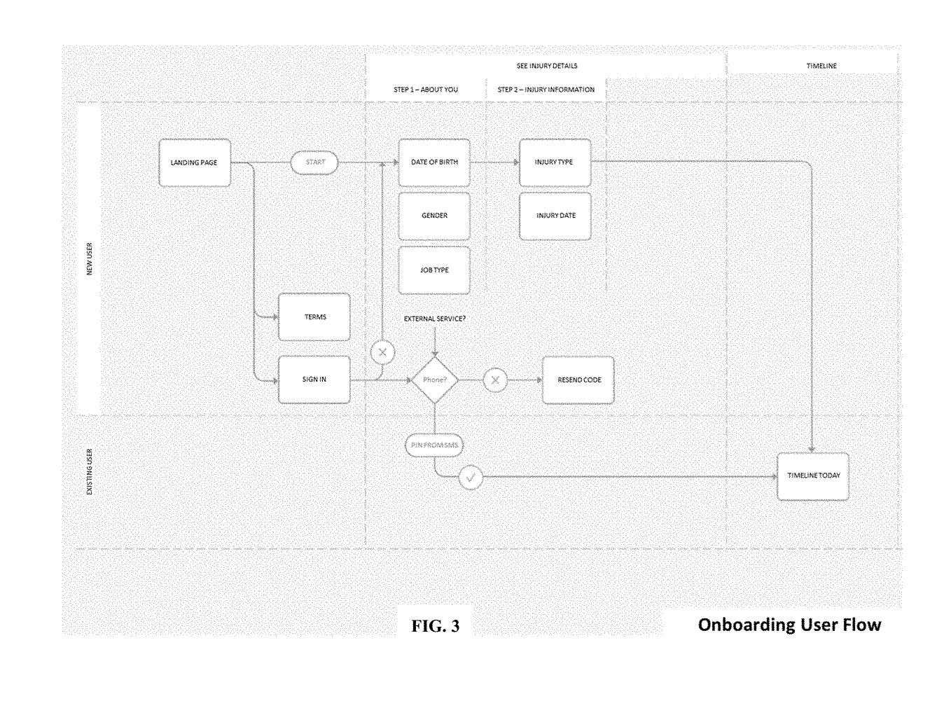

FIG. 3 shows an example schematic of the user interface for onboarding a new user or an existing user of the physical therapy program. A new user can be directed to a landing page on the application displaying the terms of usage and a start prompt. The new user can then be prompted to input personal profile information including, for example, a profile image, date of birth, gender, and job type (or occupation). After entering profile information, the user can be prompted to input injury information including, for example, injury location and injury date. Next, the user can access and track the recovery process through a recovery timeline.

An existing user can also be directed to a landing page on the application displaying the terms of usage and the start prompt. The existing user can be prompted to input login information including, for example, a username and a password. If the settings are already in place, existing users can bypass inputting of personal profile information and injury information. Existing users can modify profile information and injury information at any time. User can access the physical therapy algorithm through various devices including, for example, a mobile device or an external device that is communicatively-coupled to the mobile device. User security authorization can be confirmed by a confirmation code or pin sent to the mobile device by SMS (short message service) messaging.

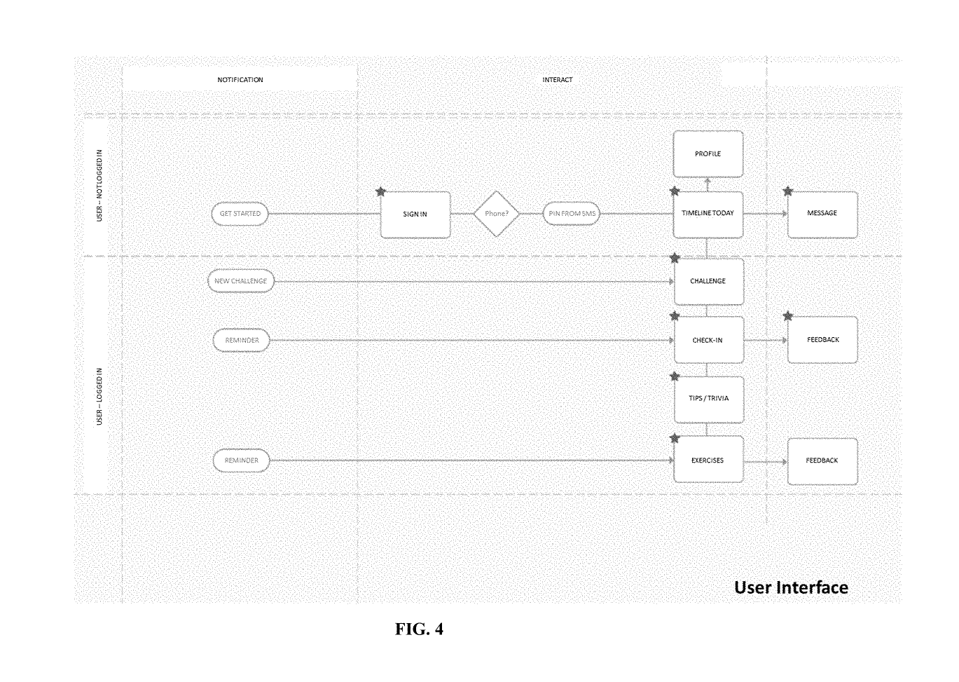

FIG. 4 shows an example schematic of the user interface for general daily user flow. A logged-in user can access various modules of the physical therapy applications on the application dashboard including, for example, challenges, trivia, and exercise recommendations.

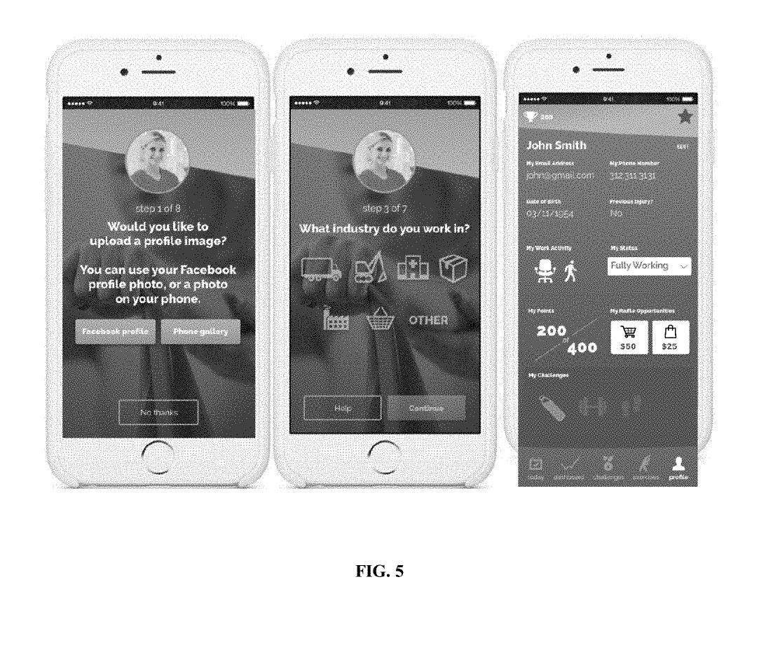

FIG. 5 shows an example display of the onboarding process. Users can choose a self image from an online source (e.g. Facebook®) or an electronic photo gallery as the profile image (left panel). Users can provide other personal information, including, for example, occupational information, injury information, and other background information to help create a customized recovery plan (center panel). The present invention can provide a simplified data gathering process to collect user information from a user without overwhelming the user. Users can access user profile information at any time to make modifications (right panel). Accordingly, the present invention can modify the recovery regimen of the user accordingly.

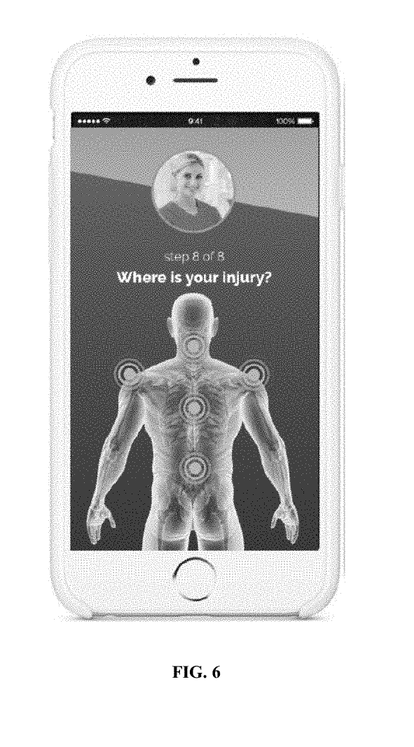

FIG. 6 shows an example display of the onboarding process. The application can display a schematic of the human body and the user can select location of injury accordingly. Users can select injury regions that can range from the neck to the back to the ankle. Visual representation of injuries can demystify data collection and increase the accuracy of injury determination.

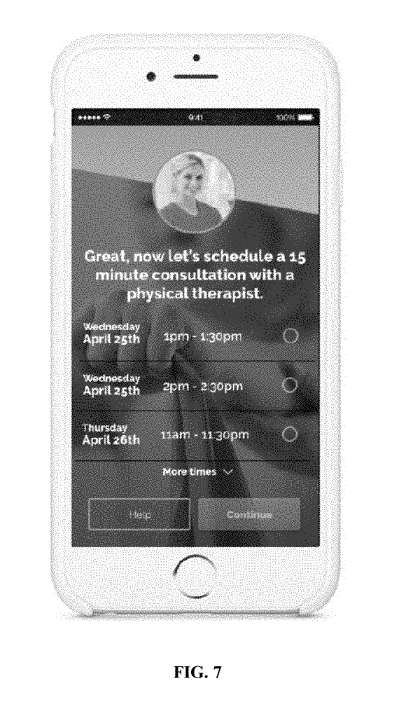

FIG. 7 shows an example display of the onboarding process. Users can schedule a virtual consultation with a physical therapist to complete diagnosis and treatment recommendations. Users can select available appointments in the immediate future, for example, within the next few days or within the next few weeks. The present invention can provide convenient methods of scheduling consultations with physical therapists that can increase likelihood of signup completion and diagnosis accuracy.

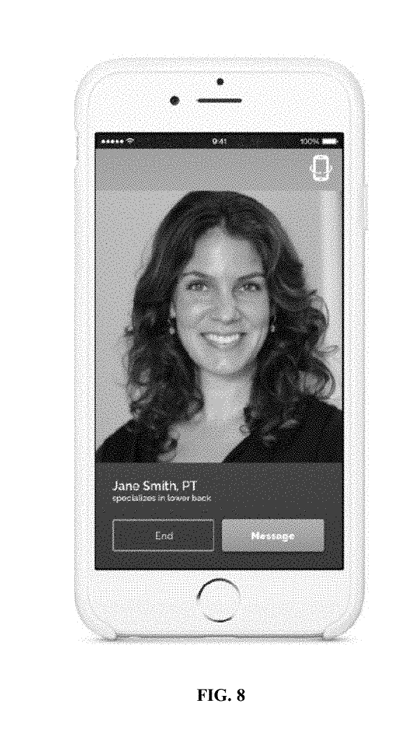

FIG. 8 shows an example display of the onboarding process. Users can conduct appointments with physical therapists via the camera on a mobile device to diagnose injuries and create recovery plans. Users can utilize video conferencing functionalities to conduct one-on-one consultation sessions with a virtual physical therapy specialist.

### Example 6: Communication Interface of the Physical Therapy Program

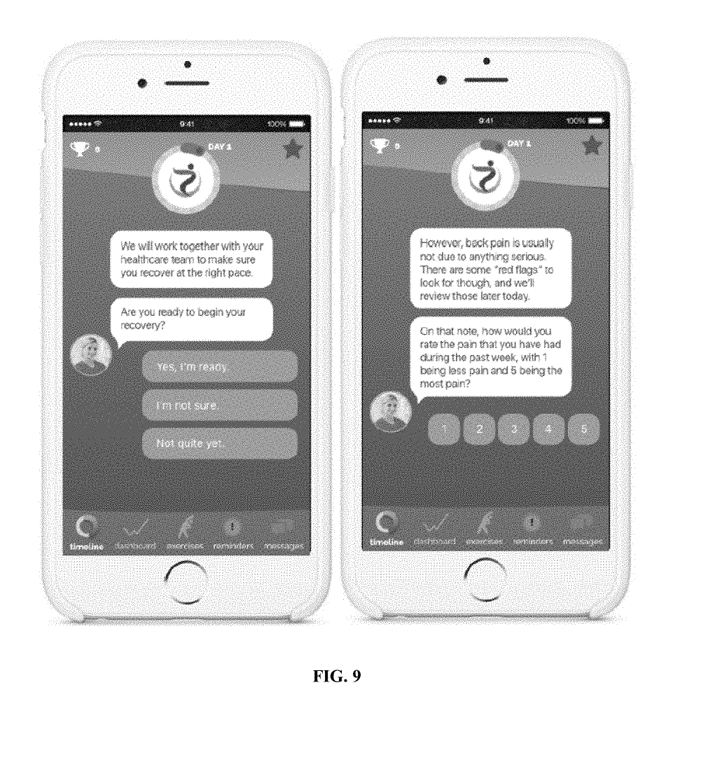

FIG. 9 shows example displays of a communication interface between the user and a virtual AI physical therapy specialist. Users can communicate with a specialist via a Conversational User Interface (CUI) to deliver a recovery program that provides a simple communication method to drive user engagement and result in effective recovery plans (left panel). A series of AI-driven experts can deliver content and recommendations through SMS messenging. The content can be accessed by professionals to provide users with a deeper sense of support in addition to the application tracker. During the course of recovery, the present invention can drive feedback from the user to determine progress and automatically adjust the recovery plan accordingly (right panel). In some embodiments, the user progress and recovery plan can be communicated to third party devices, including, for example, human resources and health professionals.

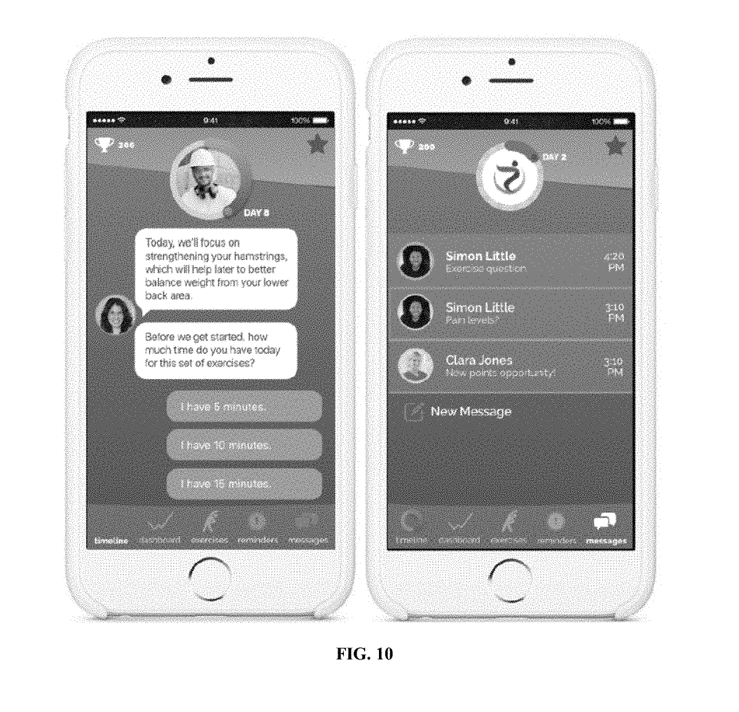

FIG. 10 shows an example display of a communication interface between the user and a virtual AI-driven physical therapy specialist. Users can communicate with the physical therapist using the present invention by the CUI. The internal messaging system can simplify communication and centralize patient information and recovery plan for physical therapists and users. Presequenced messages can help provide motivation and drive engagement with users.

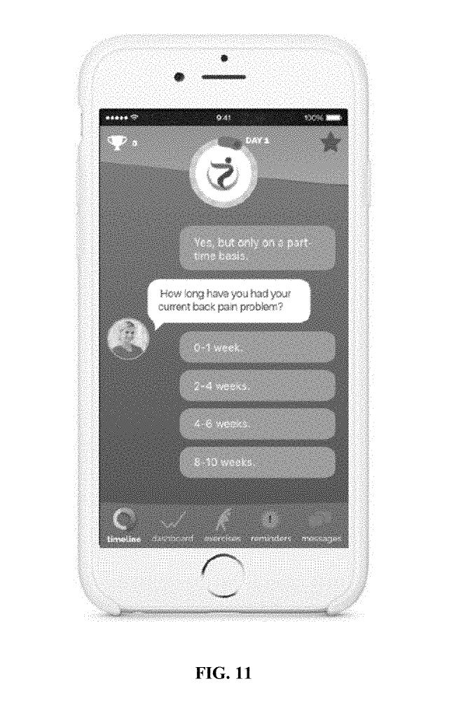

FIG. 11 shows an example display of communication interface between the user and a virtual physical therapy specialist. The CUI can use progressive data collection to learn more about a user over time can simplify the onboarding process and effectively track changes of a user's condition. AI specialists can regularly interact with a user to learn small amounts of information that can accumulate over time. Changes in user responses can guide recovery algorithms and corresponding recommendations.

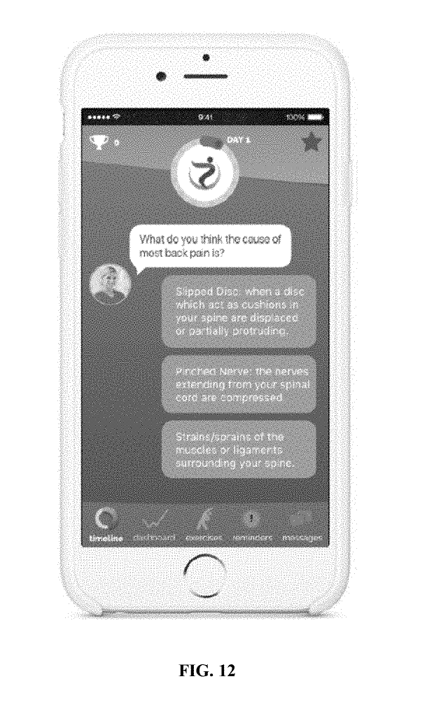

FIG. 12 shows an example display of a communication interface between the user and a virtual physical therapy specialist. During the course of recovery, the present invention can provide a user with ongoing education about the nature of the user's injury to provide an engaging dimension of treatment in addition to basic physical therapy recommendations. The present invention can provide interactive educational content tailored to a specific injury. The present invention can further provide users with a rewards system to drive user engagement.

### Example 7: Physical Therapy Program Dashboard

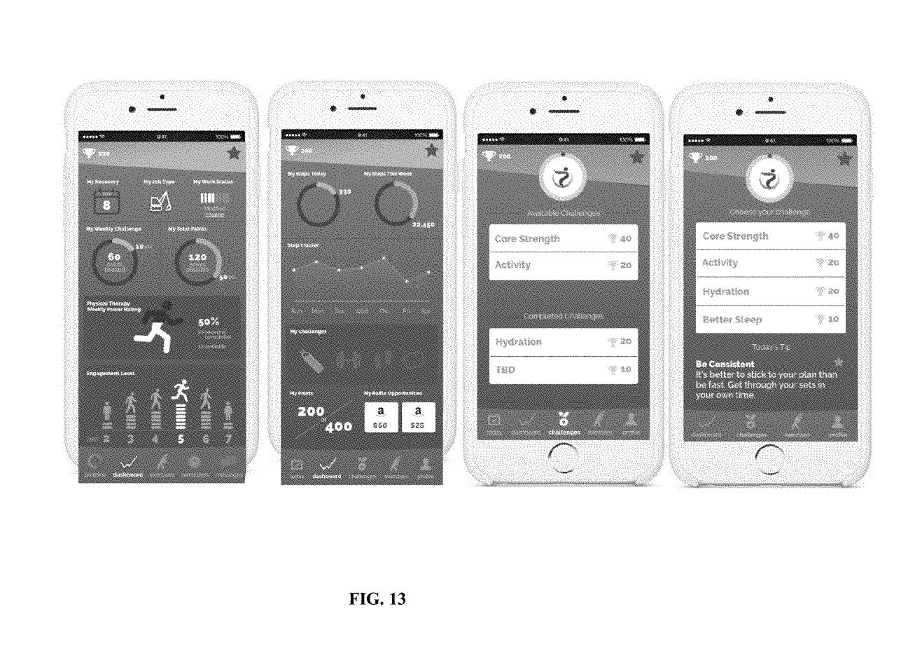

FIG. 13 shows example displays of the user dashboard that summarizes a user's recovery process to provide an ongoing positive reinforcement resource. Users can access a personalized dashboard that utilizes activity data to track user efforts and goals (first panel). The dashboard can further include challenge opportunities and a rewards system (second panel). Challenge tracking can allow initiation of available challenges. Rewards points tracking can include number of points acquired and number of points available for raffle prizes. Users can also access a persistent challenge area, where available and completed challenges can be tracked (third panel). Each available challenge can be initiated and added to the daily feed. Occasionally, the application can display a menu of available challenges to maintain user engagement and motivation (fourth panel). Users can initiate available challenges during a challenge surfacing period.

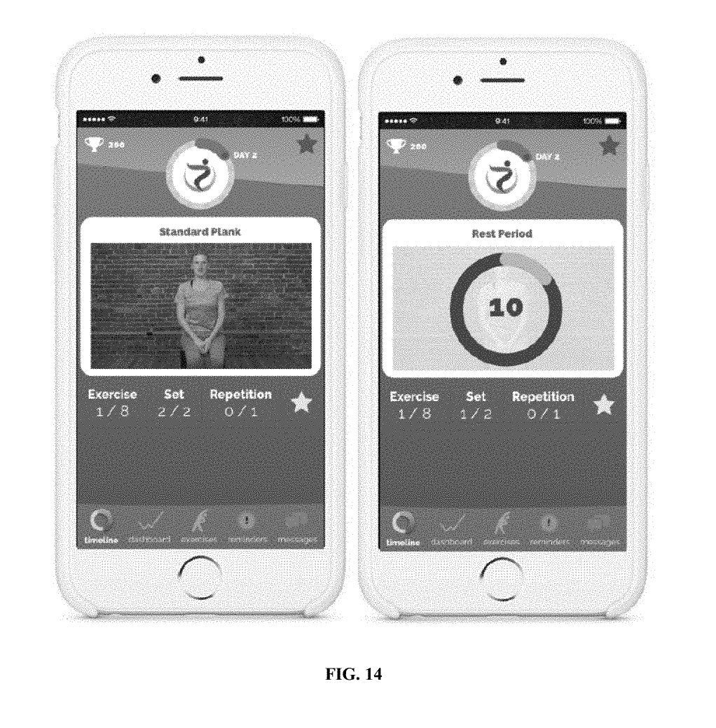

FIG. 14 shows an example display of a video-based exercise regimen. Regimens can include built-in rest/recovery periods that can help maintain user safety and maintain strong recover times.

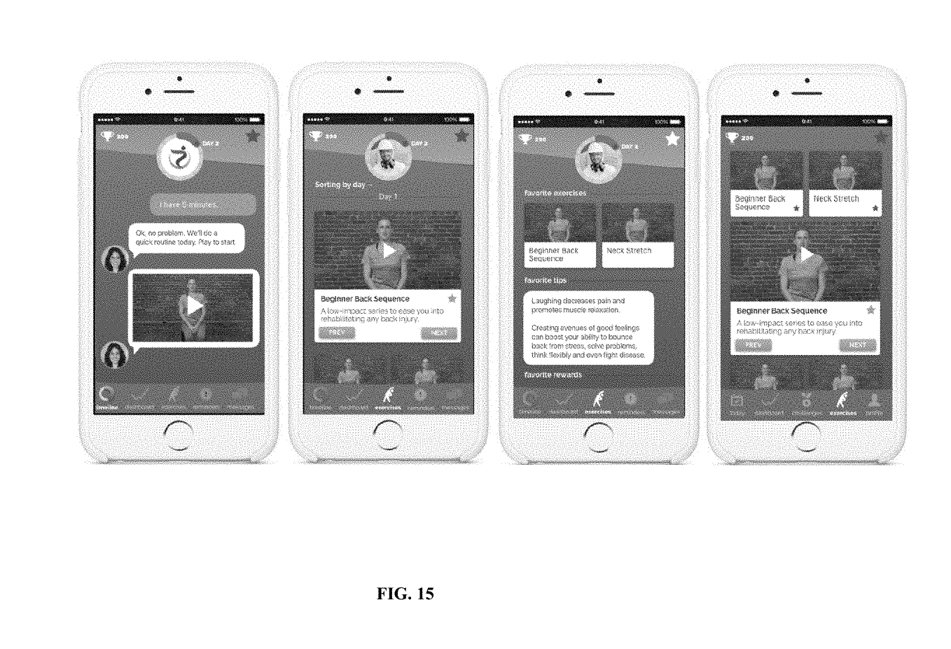

FIG. 15 shows an example display of a video-based exercise regimen communicated to the user by CUI. The AI physical therapy specialist can tailor each exercise routine to the amount of time is available. The AI physical therapy specialist can prompt the user to input their daily availability using CUI (first panel). Aligning the duration of an exercise routine can help drive continued participation and recovery success rate. Users can access any exercise in a recovery plan at any time. The exercise can be organized, for example, by the day, area of exercise, and nature of injury. Users can access and sort a library of exercise videos according to a recovery plan (second, third, and fourth panels). Users can favorite any content element by a favoriting module within the application, and access the element by type. A favoriting module can allow users to collect content and teach the application which contents are most important to the user.

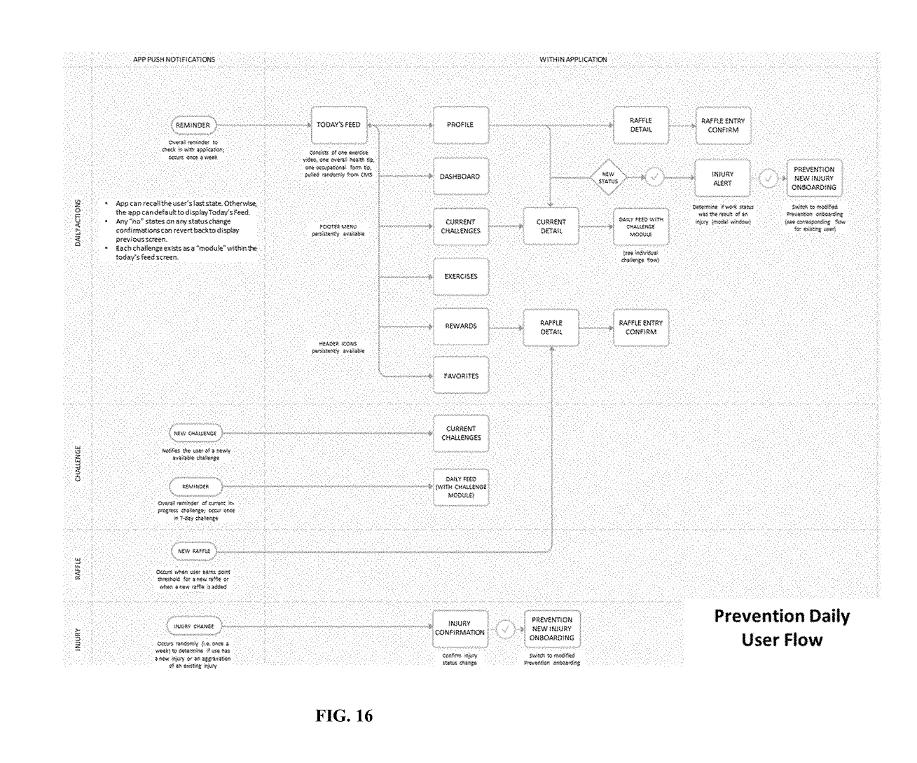

FIG. 16 shows a schematic of the user interface for prevention user flow. The physical therapy application can prompt the user with daily reminders to keep up to date with the physical therapy regimen. The daily feed can include, for example, video exercise programs, challenges, general health recommendations, and occupational health recommendations. In some embodiments, the user can modify injury information.

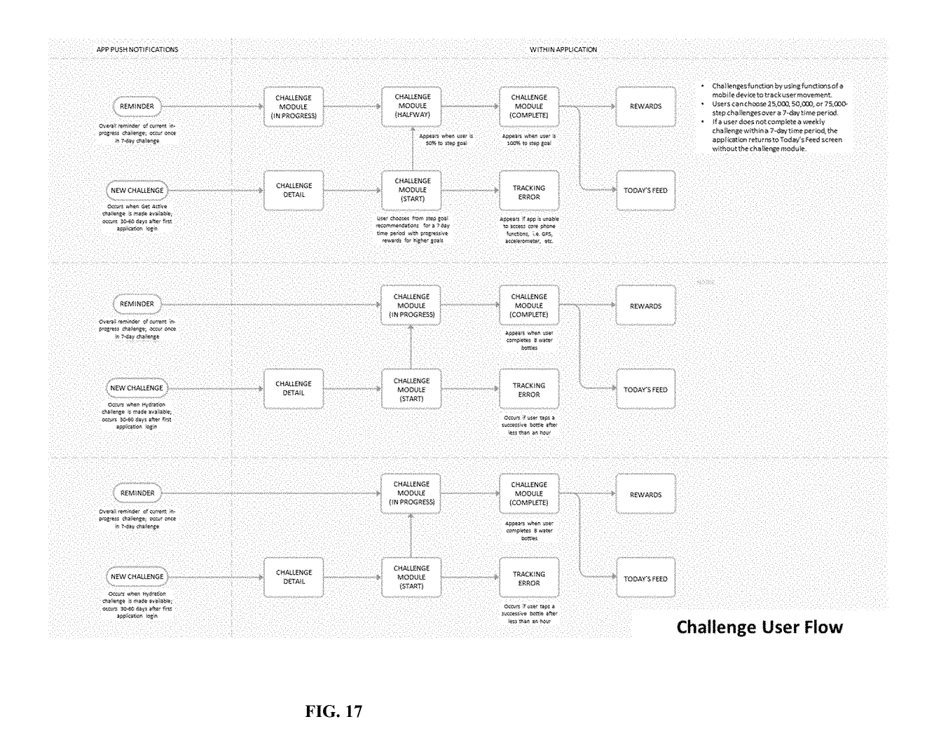

FIG. 17 shows a schematic of the user interface for challenge user flow. The application can present various challenge opportunities to the user and provide a rewards system for completing of challenges.

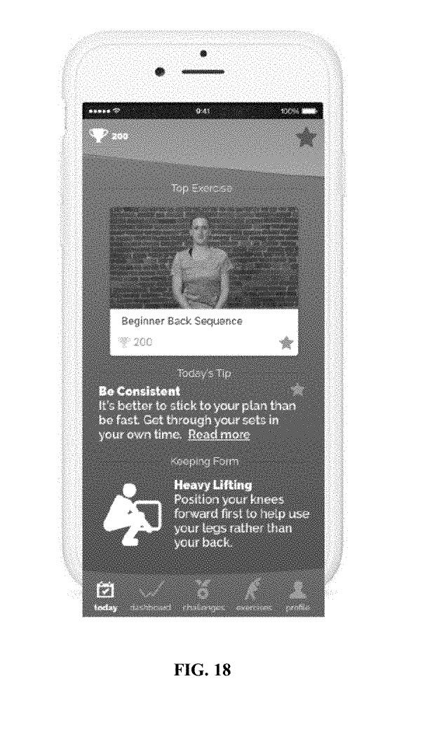

FIG. 18 shows example displays of a challenge. A therapy recommendation can be directed towards injury prevention by focusing on a daily content feed including, for example, an exercise video, a healthy lifestyle tip, and a customized occupational tip based on the user's occupation. The daily content feed can be tailored to the user's needs. The user can bookmark, by the favorite module, specific content directly from the daily feed.

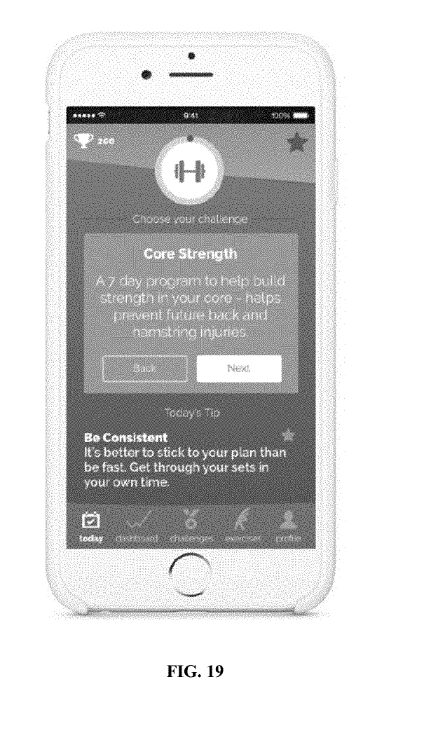

FIG. 19 shows an example display of a challenge. The Core Strength challenge can motivate users to interact with a 7-day exercise program focused on improving overall core strength. During the challenge, the application can display video content, for example, instructional exercises and the exercise recommendations. The application can further track completion of exercises and progress.

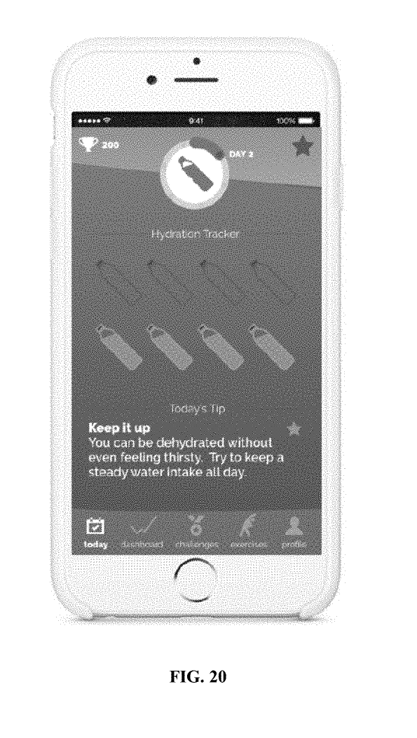

FIG. 20 shows an example display of a challenge. The Hydration challenge can motivate users to stay hydrated throughout the day. The challenge can track progress by tapping each 16 ounce water bottle icon as the user hydrates. The application can provide a visual guide for users to track hydration level. The application can display an ideal amount of daily water intake in the daily feed, and provide guidance in cases of under- or over-hydration. Rules can limit the user's ability to tap bottles throughout the day to restrict any possibility of cheating.

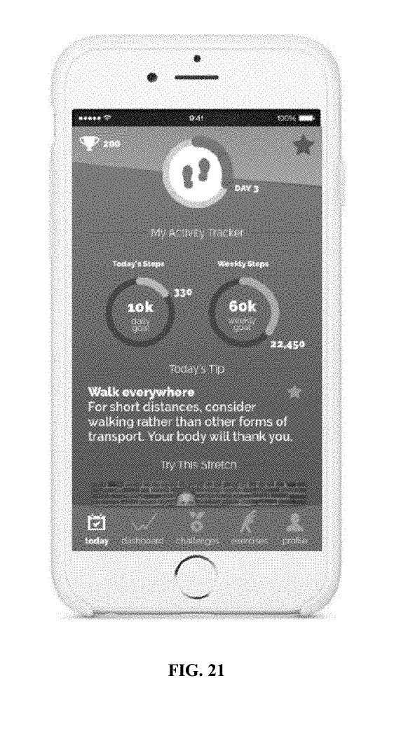

FIG. 21 shows an example display of a challenge. The Activity challenge can motivate users to stay active throughout the day by providing daily and weekly step count goals. A tracker module can be displayed in the daily feed to track the user's steps progress. The step tracker can utilize the mobile device's accelerometer and GPS mapping to determine user daily movement and translate movements into steps. The tracker can automatically set goals for each user based on overall activity recommendation and user's activity history.

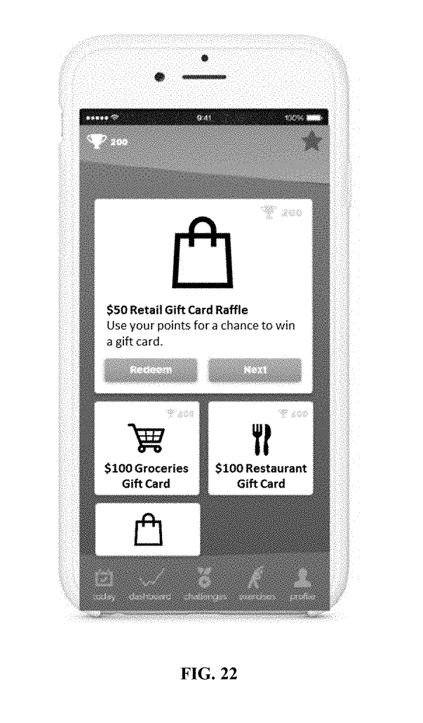

FIG. 22 shows an example display of available raffles based on a rewards system. Users can enter a variety of raffles using activity points earned through engagement with the application. Raffle prizes can include, for example, retail coupons and giftcards.

### Example 8: Data Segmentation

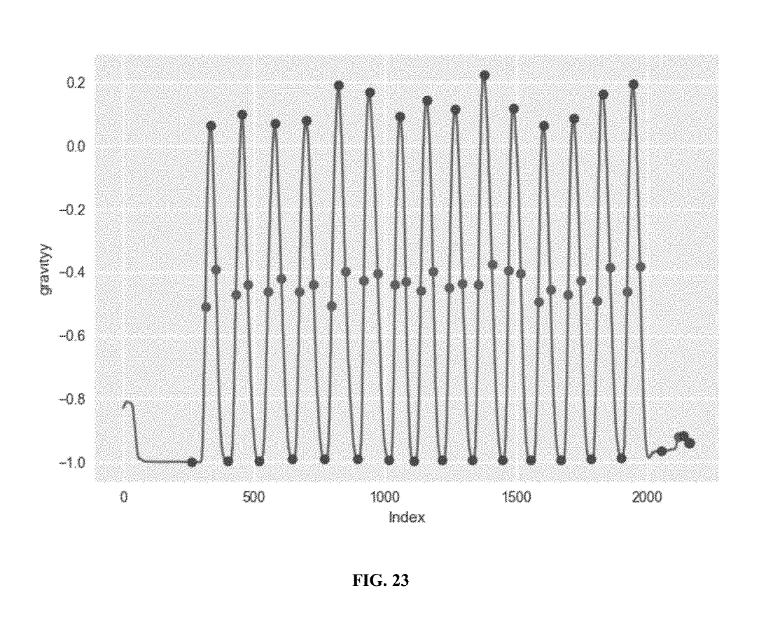

A critical step of interpreting continuous motion data is identification of the beginning, midpoint, and end of a repetitive motion of an exercise. A logistic regression model, which uses the mean value of the y-axis accelerometer data (gravity y), was used to verify the orientation of a mobile device during an arm raise exercise. The mobile device served as the motion tracking device. The static orientation of a mobile device prior to starting the exercise was with the subject's arm straight down by the waist. FIG. 23 illustrates a waveform motion data produced from the y-axis accelerometer data during the course of the exercise (index). As shown in FIG. 23, the model accurately identified the beginning, midpoint, and end of each repetition of the exercise. The beginning and end points are denoted by the black dots at gravity of approximately −1.0 (troughs). The midpoint is denoted by the black dots at gravity of approximately between 0.0-0.2 (crests).

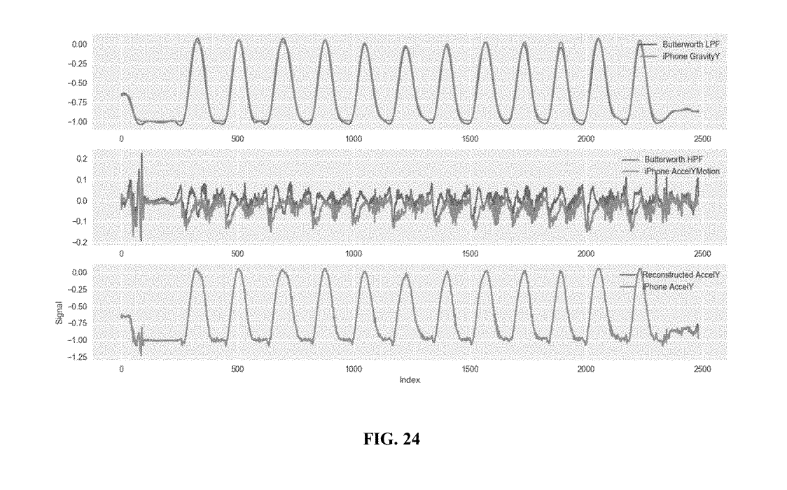

The accelerometer data gathered by the mobile device (iPhone®) was filtered to generate smoother and more consistent data. FIG. 24 illustrates a waveform motion data produced from the y-axis accelerometer data during the course of the exercise (index) after filtering. A 5^{th} order Butterworth HP/LP filter with a cutoff frequency of 0.7 Hz was used.

### Example 9: Training Models and Feature Selection

After segmenting the continuous motion data into individual repetitions, the data was analyzed to discriminate between correct and incorrect motion. In a typical physical therapy setting in which a subject is performing an exercise, a physical therapist observes the subject and provides guidance regarding adequacy of range, speed, and positioning of the movements. In an arm raise exercise, for example, the physical therapist can observe the subject's movements to ensure that the arm is raised to an adequate height, e.g. 90 degrees from the horizontal plane, but not too high, e.g. greater than 90 degrees from the horizontal plane. The physical therapist can observe the subject's movements to ensure that the positioning is adequate, e.g. the arm is not pronated or supinated during the routine, and the elbow is fully extended and not bent. The physical therapist can further observe the subject's movements to ensure that the speed is adequate, e.g. the arm is not raised faster than once every 2 seconds. To mimic the exercise assessment by a physical therapist, training models were used to model correct and incorrect movements and positioning.

A single-joint exercise of the shoulder flexion was used as an example to implement the training models. The following parameters constituted a “correct” performance:
1. Start with arms down at hips with thumb up and forward;
2. Extend arm upward 90 degrees from the horizontal plane such that the arm extends straight out.
3. Bring arm back down at the same motion and speed.
4. Each repetition is no greater than 3 seconds and no less than 1 second.

The following parameters constituted an “incorrect” performance:
1. Each repetition is greater than 3 seconds or less than 1 second.
2. Palm down during exercise.
3. Palm up during exercise.
4. Pronate arm from thumb up to palm down.
5. Raise hand across body.
6. Raise outward away from torso.
7. Extend arm upward beyond 90 degrees or less than 45 degrees.
8. Extend arm upward backwards beyond baseline point (0 degrees).
9. Elevate arm with elbow bent.

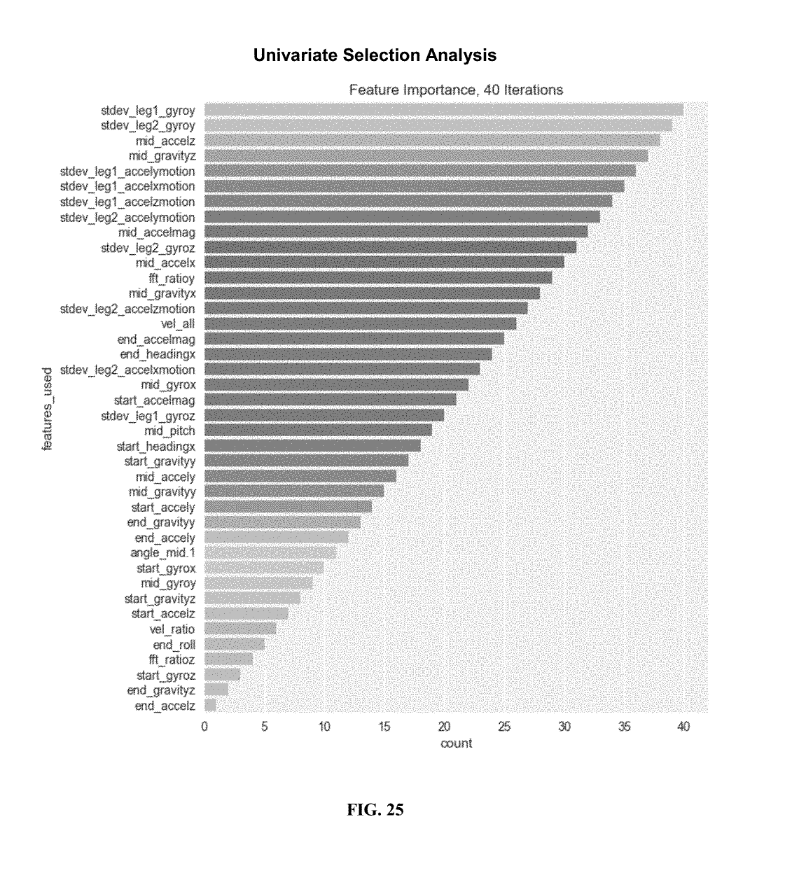

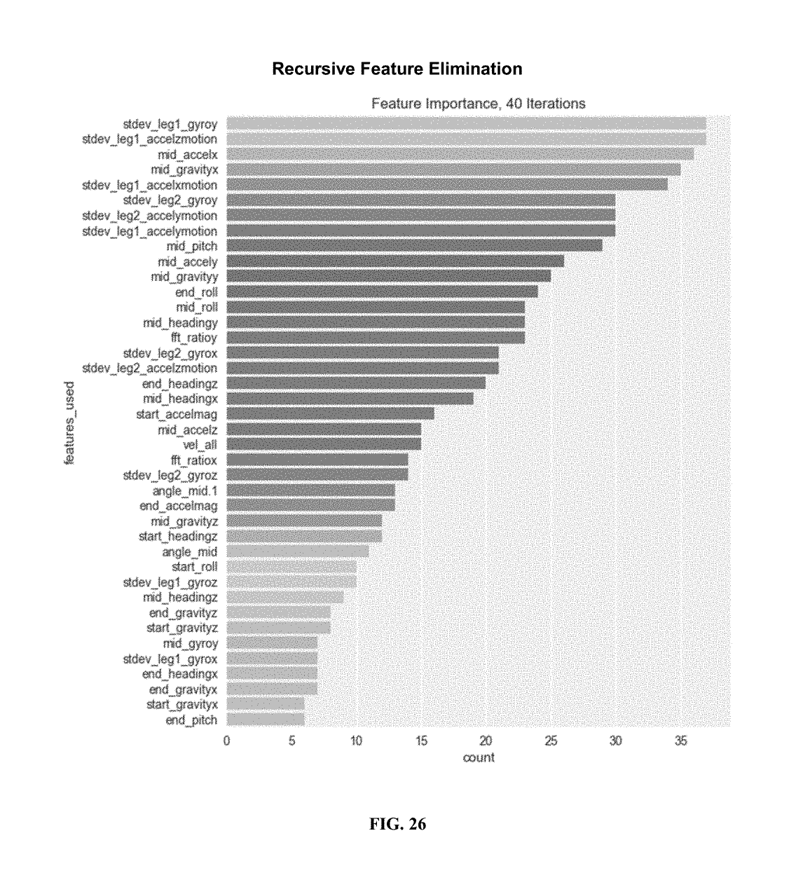

Using the guidelines above, motion data was collected from a user performing the shoulder raise exercise both correctly (20 sets of 20 repetitions) and incorrectly (20 sets of 20 repetition). A binary classification of whether a particular repetition was performed correctly or incorrectly was determined. The specific type of error made by the user was not further classified. As listed in FIGS. 25-27, 40 different motion descriptors (features) were used in this classification. These features included information regarding the three-dimensional acceleration, velocity, and orientation of the motion tracking device (iPhone® mobile device) attached to the subject during the exercise. Data was collected from both the accelerometer and the gyroscope of the motion tracking device.

Not all features are equally important in evaluating whether the exercise is correctly performed. For example, for an exercise which involves rotation about the X-axis, data which describes motion around the X-axis would be more informative than data regarding rotation about the Y- and Z-axes. Nevertheless, Y- and Z-axis data can still be useful, for example, to detect erroneous motion. To distinguish which features are most significant in the case of a straight arm shoulder flexion exercise, feature importance was ranked using three methods: Univariate Selection Analysis (SelectK), Recursive Feature Elimination (RFE), and Principal Component Analysis (PCA) are shown in FIGS. 25-27, respectively. The relative importance of the 40 features using each of the three methods is shown. In each analysis, cross validation was performed on classification models containing the 40 features. The counts for the features selected at each iteration are shown.

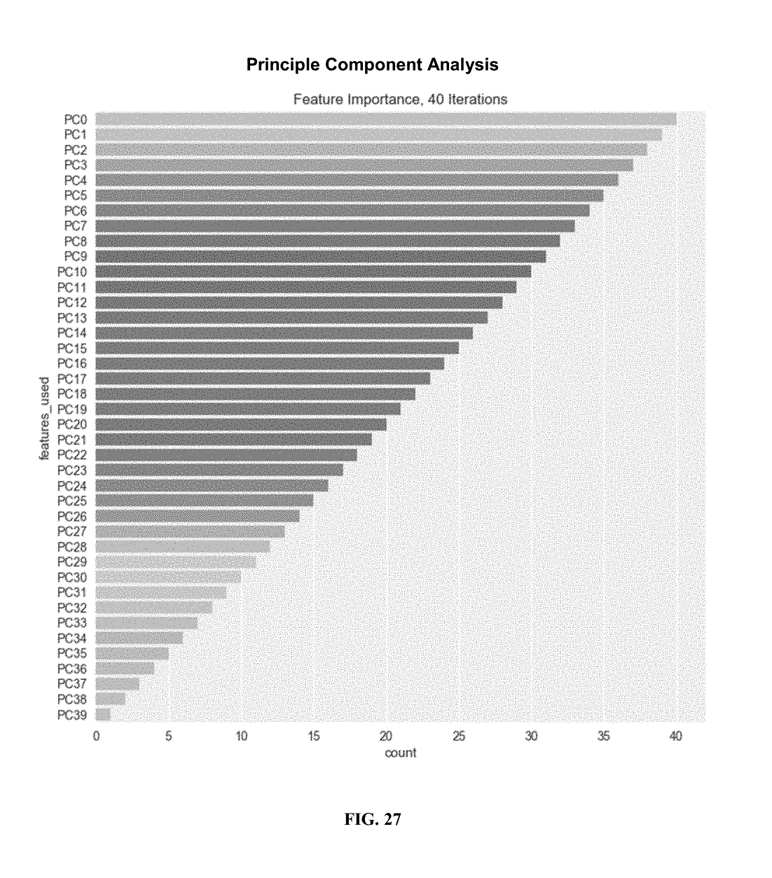

The most important feature was stdev_leg1_gyroy, corresponding to the standard deviation of the gyroscope data around y-axis for the first part (leg 1) of the segmented exercise. Labels for the PCA shown in FIG. 27 were numerical (PC0-PC39) because PCA evaluates the entire feature space to compute which components contribute to the most variance in the data.

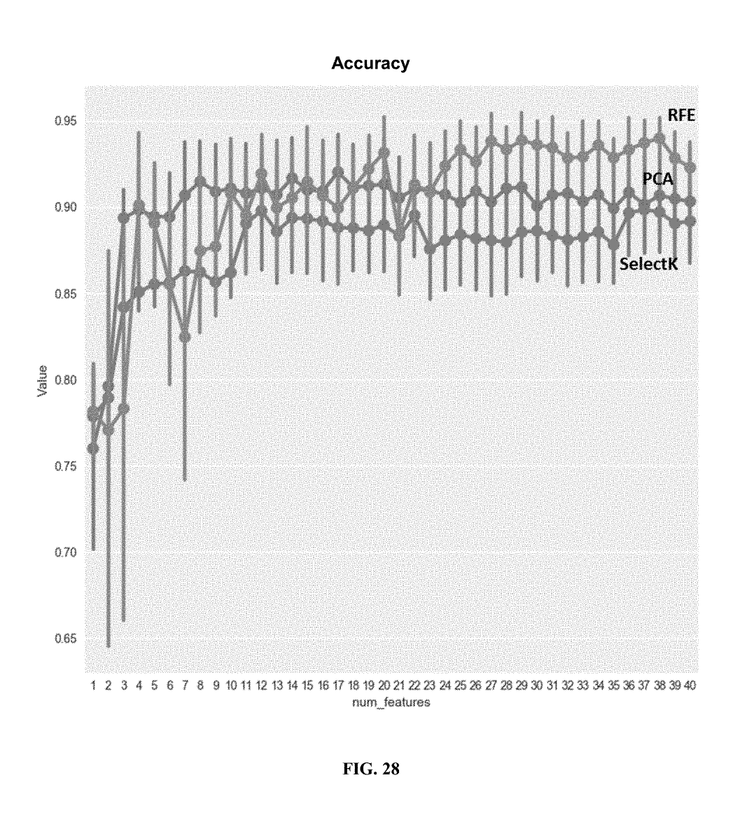

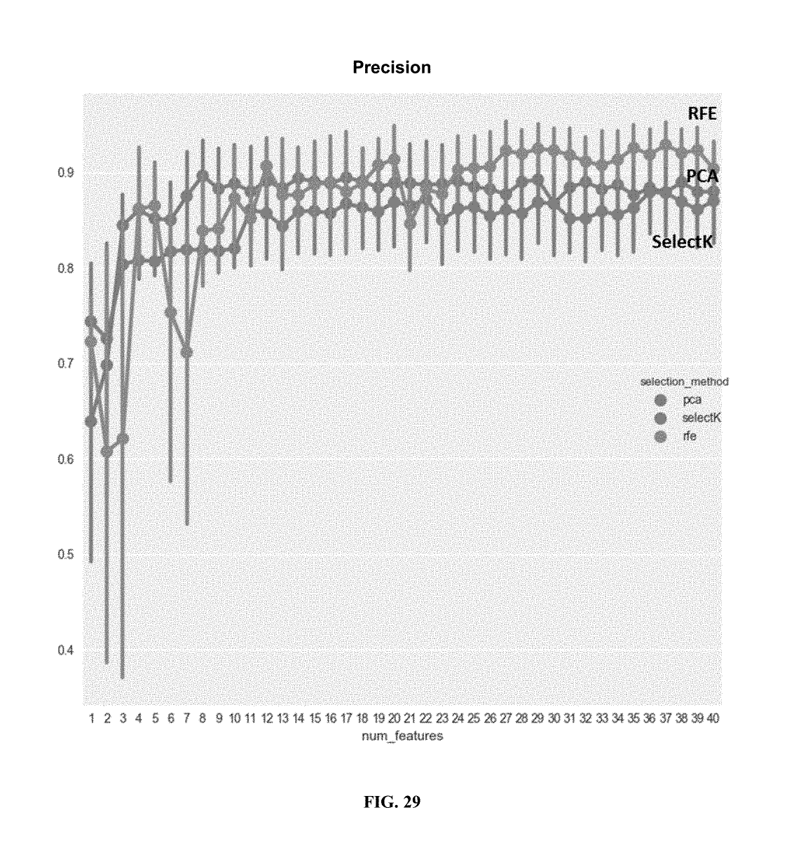

The accuracy (FIG. 28) and precision (FIG. 29) of each classification were relatively poor when only a single feature was considered in the analysis. Classification performance improved as more features were considered in the analysis, and leveled off with approximately 15-20 features. Overall, feature selection with RFE tended to produce the best results, and selecting the top 20 most significant features provided a reasonable balance between accuracy and overfitting.

### Example 10: Classification Performance

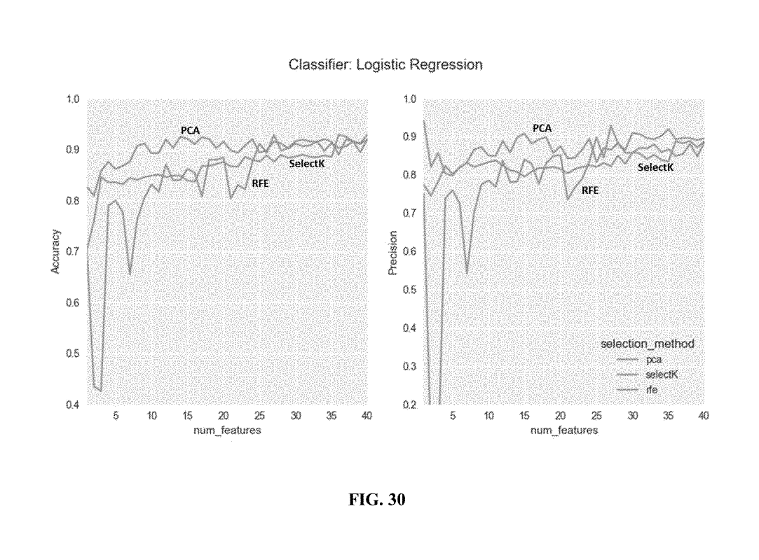

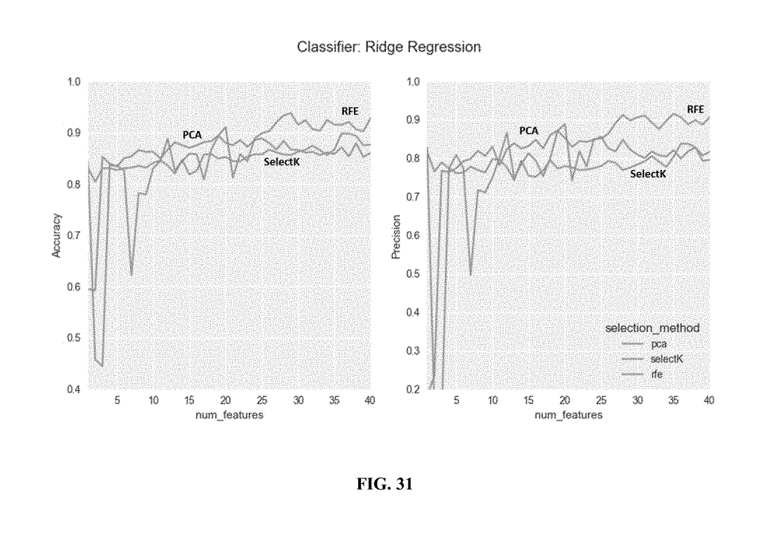

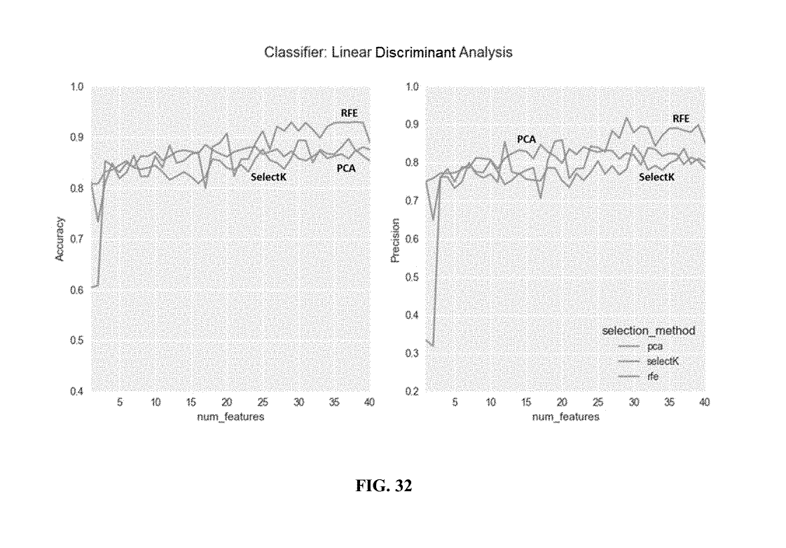

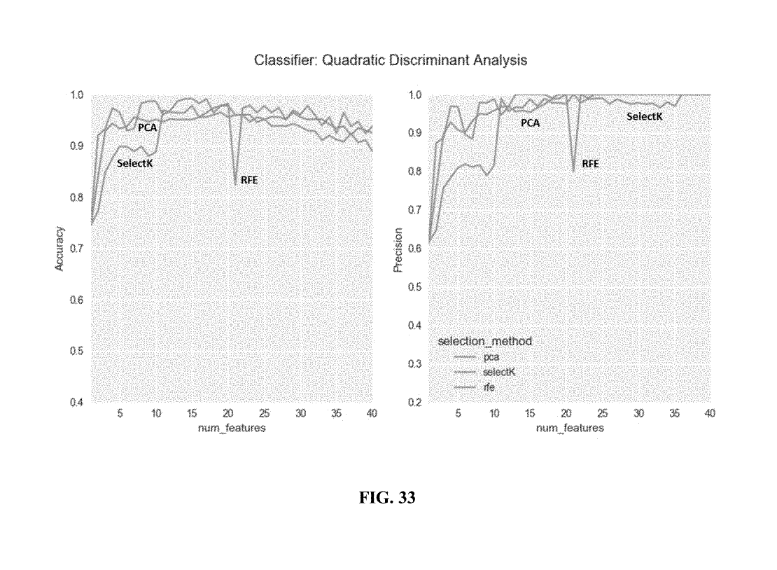

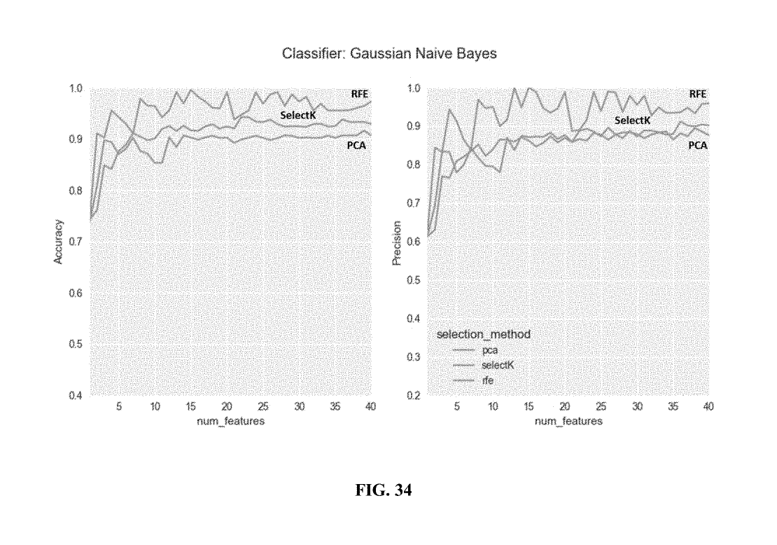

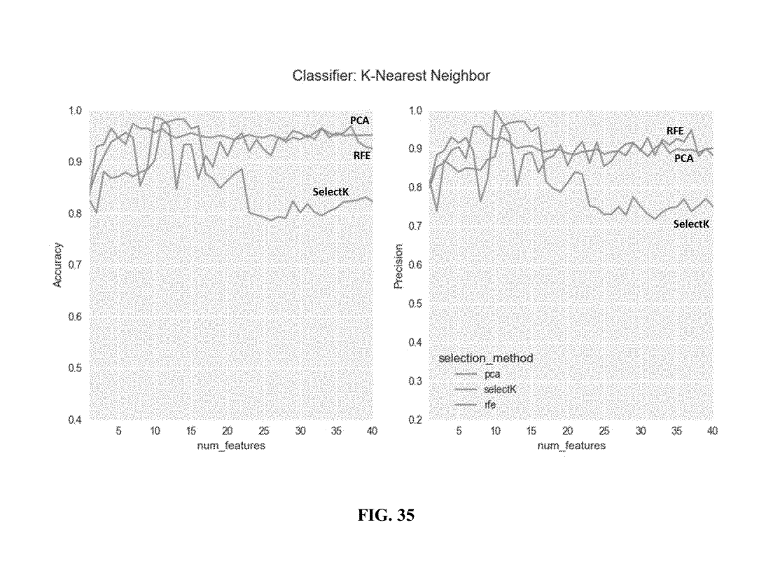

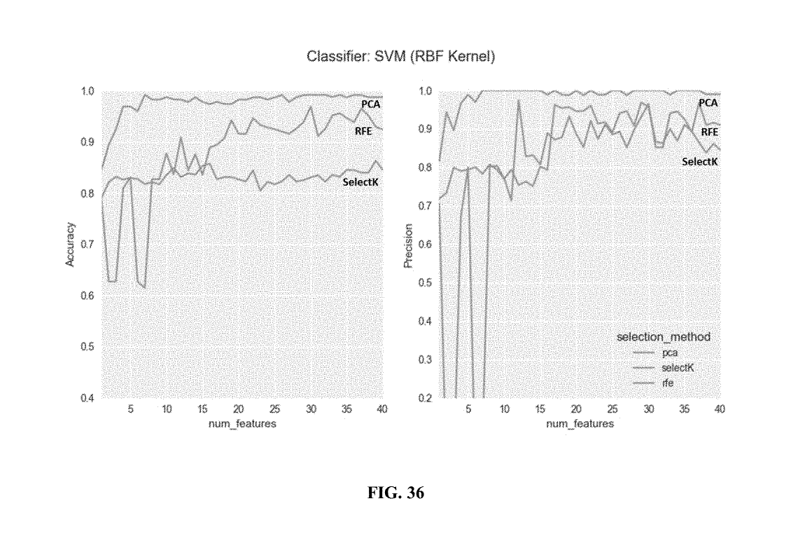

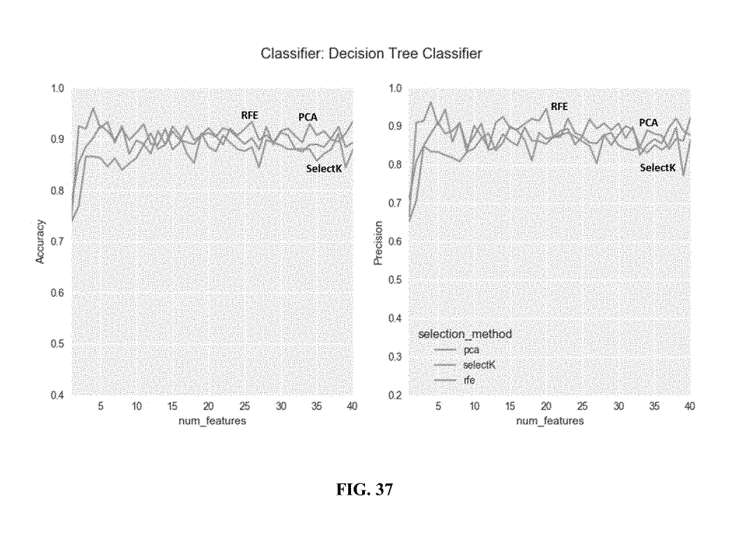

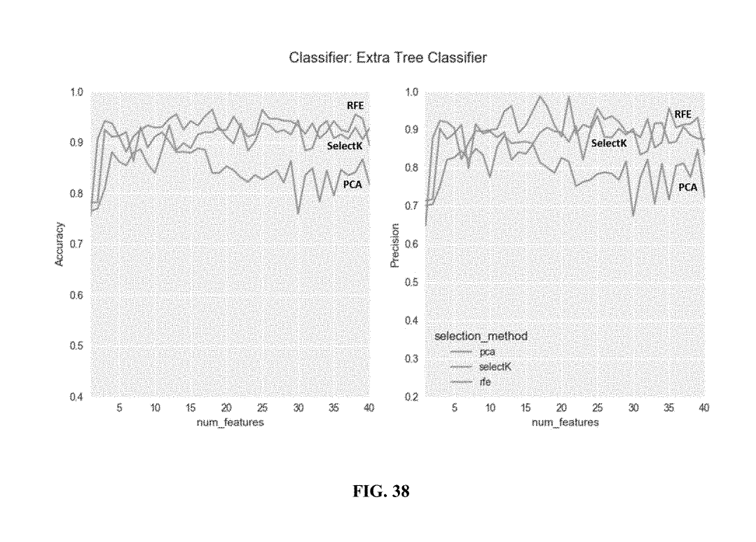

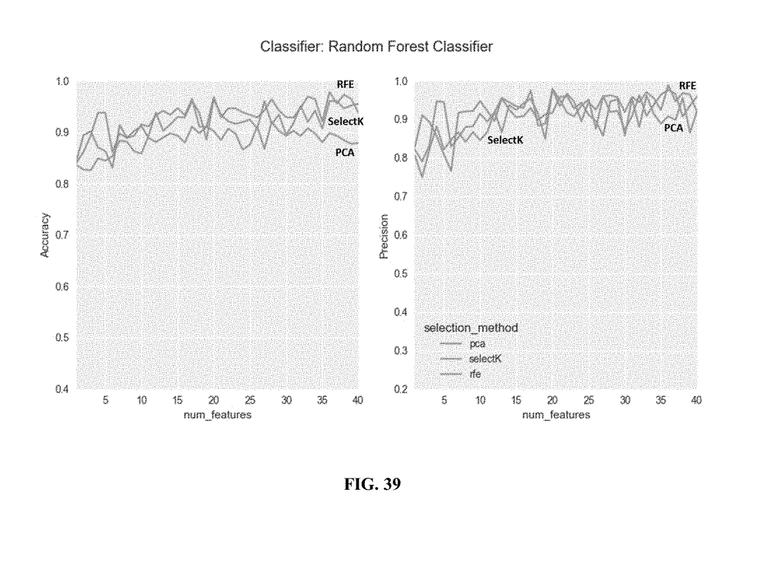

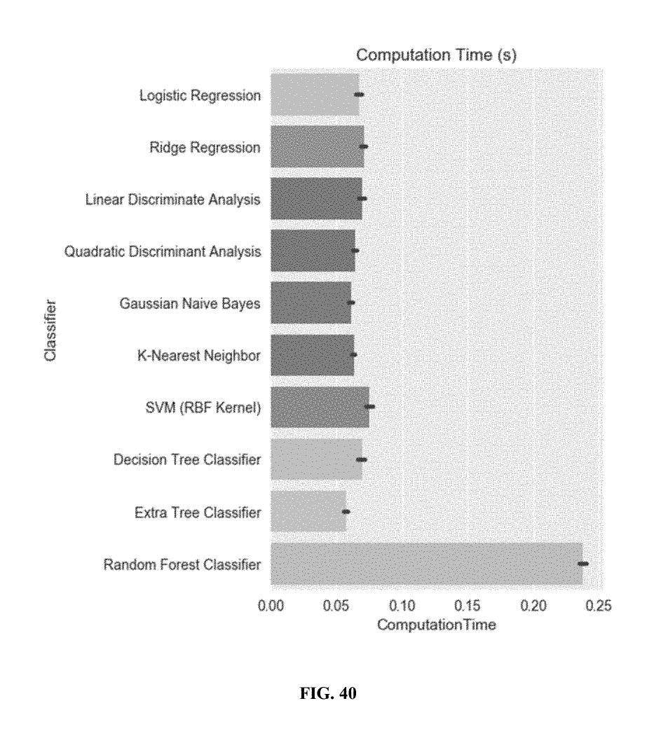

Commonly used classification strategies (listed on TABLE 1) were used to study relative performance for the classification task described in Example 9. For each classifier, the features were ranked from most important to least important using the methods described, and classification performance were evaluated. The graphs shown in FIGS. 30-39 demonstrate the precision and accuracy of each classifier as a function of the number of the most important features. Because performance tended to level off after the top 20 features were included, the top 20 features were used to directly compare all 10 classifiers, as summarized in TABLE 1. Recursive feature elimination was used as the feature selection method. The results indicate that, with the top 20 features ranked by RFE, the highest accuracy and precision were achieved by Quadratic Discriminant Analysis (QDA), Gaussian Naive Bayes (GNB), and Random Forest Classifier (RFC). With respect to computation time as shown in FIG. 40, QDA and GNB significantly outperformed RFC, which suggests that QDA and GNB are most suitable for real-time applications.

TABLE 1

| | | | Computation |
|---|---|---|---|
| Classifier | Accuracy | Precision | Time (s) |
| Logistic Regression | 0.8841 | 0.8519 | 0.0703 |
| Ridge Regression | 0.9106 | 0.8888 | 0.0801 |
| Linear Discriminant Analysis | 0.9073 | 0.8589 | 0.0746 |
| Quadratic Discriminant Analysis | 0.9822 | 1.0000 | 0.0677 |
| Gaussian Naive Bayes | 0.9911 | 0.9889 | 0.0649 |
| K-Nearest Neighbor | 0.9107 | 0.8561 | 0.0678 |
| SVM (RBF Kernel) | 0.9158 | 0.8866 | 0.0854 |
| Decision Tree Classifier | 0.9210 | 0.9450 | 0.0796 |
| Extra Tree Classifier | 0.9241 | 0.8811 | 0.0705 |
| Random Forest Classifier | 0.9686 | 0.9764 | 0.2602 |

### EMBODIMENTS

### Embodiment 1

A method for interpreting an exercise movement performed by a subject, the method comprising: a) receiving motion data from a motion tracking device based on the exercise movement performed by the subject; b) interpreting the motion data by the motion tracking device using a machine learning algorithm; c) determining by the motion tracking device whether the subject performed the exercise movement correctly based on a reference model of the exercise movement; and d) providing by the motion tracking device a recommendation to the subject to increase the accuracy of the exercise movement performed by the subject based on the reference model of the exercise movement.

### Embodiment 2

The method of embodiment 1, wherein the exercise movement is a multiaxial movement.

### Embodiment 3

The method of embodiment 1 or 2, wherein determining whether the subject performed the exercise movement correctly based on a reference model of the exercise movement is based on the speed and orientation of the movement performed by the subject.

### Embodiment 4

The method of any one of embodiments 1-3, wherein the motion tracking device is attached to the subject.

### Embodiment 5

The method of any one of embodiments 1-3, wherein the motion tracking device is attached to an exercise machine on which the subject performs the exercise movement.

### Embodiment 6

The method of any one of embodiments 1-5, wherein the recommendation is a visual feedback.

### Embodiment 7

The method of any one of embodiments 1-6, wherein the recommendation is an audio feedback.

### Embodiment 8

The method of any one of embodiments 1-7, wherein the recommendation is a haptic feedback.

### Embodiment 9

The method of any one of embodiments 1-8, further comprising calibrating by the motion tracking device the exercise movement performed by the subject to the reference model of the exercise movement based on a baseline orientation of the motion tracking device and the motion data of the exercise movement performed by the subject.

### Embodiment 10

The method of embodiment 9, wherein the calibrating is performed by machine learning.

### Embodiment 11

The method of any one of embodiments 1-10, further comprising modifying by the motion tracking device the reference model of the exercise movement based on the motion data of the exercise movement performed by the subject.

### Embodiment 12

The method of any one of embodiments 1-11, wherein the interpreting, the determining, and the providing are additionally performed by a server that is communicatively coupled to the motion tracking device.

### Embodiment 13

A computer program product comprising a non-transitory computer-readable medium having computer-executable code encoded therein, the computer-executable code adapted to be executed to implement a method for interpreting an exercise movement performed by a subject, the method comprising: a) providing a physical therapy guidance system, wherein the physical therapy guidance system comprises: i) a data storage medium; ii) a detection module; iii) an accelerometer module; iv) a gyroscope module; v) a search module; vi) an interpretation module; and vii) an output module; b) storing by the data storage medium a reference model of the exercise movement; c) detecting by the detection module the exercise movement performed by the subject; d) searching by the search module the reference model of the exercise movement based on the exercise movement performed by the subject; e) measuring by the accelerometer module a speed of the exercise movement performed by the subject relative to the reference model of the exercise movement; f) measuring by the gyroscope module an orientation of the exercise movement performed by the subject relative to the reference model of the exercise movement; g) determining by the interpretation module whether the subject performed the exercise movement correctly based on the reference model of the exercise movement using machine learning; and h) outputting by the output module a recommendation based on the accuracy of the exercise movement performed by the subject.

### Embodiment 14

The computer program product of embodiment 13, wherein the physical therapy guidance system further comprises a display module and the method further comprises displaying by the display module the reference model of the exercise movement based on the speed and orientation of the exercise movement performed by the subject.

### Embodiment 15

The computer program product of embodiment 13 or 14, wherein the physical therapy guidance system further comprises a calibration module and the method further comprises calibrating by the calibration module the exercise movement performed by the subject based on a baseline orientation of the motion tracking device, and the speed and orientation of the exercise movement performed by the subject.

### Embodiment 16

The computer program product of any one of embodiments 13-15, wherein the physical therapy guidance system further comprises a voice recognition module and the method further comprises recognizing by the voice recognition module a voice command.

What is claimed is:

1. A method for interpreting an exercise movement performed by a subject, the method comprising:
a) receiving motion data from a motion tracking device based on the exercise movement performed by the subject;
b) interpreting the motion data by the motion tracking device using a machine learning algorithm;
c) determining by the motion tracking device whether the subject performed the exercise movement correctly based on a reference model of the exercise movement; and
d) providing by the motion tracking device a recommendation to the subject to increase the accuracy of the exercise movement performed by the subject based on the reference model of the exercise movement.

2. The method of claim 1, wherein the exercise movement is a multiaxial movement.

3. The method of claim 1, wherein determining whether the subject performed the exercise movement correctly based on a reference model of the exercise movement is based on the speed and orientation of the movement performed by the subject.

4. The method of claim 1, wherein the motion tracking device is attached to the subject.

5. The method of claim 1, wherein the motion tracking device is attached to an exercise machine on which the subject performs the exercise movement.

6. The method of claim 1, wherein the recommendation is a visual feedback.

7. The method of claim 1, wherein the recommendation is an audio feedback.

8. The method of claim 1, wherein the recommendation is a haptic feedback.

9. The method of claim 1, further comprising calibrating by the motion tracking device the exercise movement performed by the subject to the reference model of the exercise movement based on a baseline orientation of the motion tracking device and the motion data of the exercise movement performed by the subject.

10. The method of claim 9, wherein the calibrating is performed by machine learning.

11. The method of claim 1, further comprising modifying by the motion tracking device the reference model of the exercise movement based on the motion data of the exercise movement performed by the subject.

12. The method of claim 1, wherein the interpreting, the determining, and the providing are additionally performed by a server that is communicatively coupled to the motion tracking device.

13. A computer program product comprising a non-transitory computer-readable medium having computer-executable code encoded therein, the computer-executable code adapted to be executed to implement a method for interpreting an exercise movement performed by a subject, the method comprising:
a) providing a physical therapy guidance system, wherein the physical therapy guidance system comprises:
i) a data storage medium;
ii) a detection module;
iii) an accelerometer module;
iv) a gyroscope module;
v) a search module;
vi) an interpretation module; and
vii) an output module;
b) storing by the data storage medium a reference model of the exercise movement;
c) detecting by the detection module the exercise movement performed by the subject;
d) searching by the search module the reference model of the exercise movement based on the exercise movement performed by the subject;
e) measuring by the accelerometer module a speed of the exercise movement performed by the subject relative to the reference model of the exercise movement;
f) measuring by the gyroscope module an orientation of the exercise movement performed by the subject relative to the reference model of the exercise movement;
g) determining by the interpretation module whether the subject performed the exercise movement correctly based on the reference model of the exercise movement using machine learning; and
h) outputting by the output module a recommendation based on the accuracy of the exercise movement performed by the subject.

14. The computer program product of claim 13, wherein the physical therapy guidance system further comprises a display module and the method further comprising displaying by the display module the reference model of the exercise movement based on the speed and orientation of the exercise movement performed by the subject.

15. The computer program product of claim 13, wherein the physical therapy guidance system further comprises a calibration module and the method further comprising calibrating by the calibration module the exercise movement performed by the subject based on a baseline orientation of the motion tracking device, and the speed and orientation of the exercise movement performed by the subject.

16. The computer program product of claim 13, wherein the physical therapy guidance system further comprises a voice recognition module and the method further comprising recognizing by the voice recognition module a voice command.
